(19) United States
(12) Patent Application Publication
(10) Pub. No.: US 2013/0032791 A1
(43) Pub. Date: Feb. 7, 2013

(54) CONJUGATED POLYMERS HAVING AN IMINE GROUP AT THE INTRACHAIN ELECTRON DONOR BRIDGEHEAD POSITION USEFUL IN ELECTRONIC DEVICES

(71) Applicants: Guillermo C. Bazan, Goleta, CA (US); Jason D. Azoulay, Santa Barbara, CA (US); Bruno Caputo, Goleta, CA (US)

(72) Inventors: Guillermo C. Bazan, Goleta, CA (US); Jason D. Azoulay, Santa Barbara, CA (US); Bruno Caputo, Goleta, CA (US)

(21) Appl. No.: 13/523,716

(22) Filed: Jun. 14, 2012

Related U.S. Application Data
(60) Provisional application No. 61/501,147, filed on Jun. 24, 2011.

(51) Int. Cl. C08G 75/32; H01L 51/54; C07C 251/20; C07F 7/22; C07F 5/02; H01L 51/46; C07D 495/04

(52) U.S. Cl. 257/40; 528/7; 528/9; 549/43; 549/3; 564/8; 564/270; 257/E51.026

(57) ABSTRACT

Described herein are novel light absorbing conjugated polymeric electron donor materials for organic photovoltaic devices and other applications. In one embodiment, the polymer structure comprises a conjugated electron rich donor unit with an imine functionality at the bridgehead position and a conjugated electron deficient unit in the polymer backbone arranged in an alternating fashion. Monomers suitable for making the polymers, and devices utilizing the polymers, are also disclosed.

### CROSS-REFERENCE TO RELATED APPLICATIONS

This patent application claims priority benefit of U.S. Provisional Patent Application No. 61/501,147, filed Jun. 24, 2011. The entire contents of that application are hereby incorporated by reference herein.

### STATEMENT REGARDING FEDERALLY SPONSORED RESEARCH OR DEVELOPMENT

This invention was made with United States government support under grant no. 8-448777-22405 from the Department of Energy. The government has certain rights in the invention.

### FIELD OF THE INVENTION

The present invention relates generally to conjugated polymers for use in electronic devices, such as N-4H-cyclopenta[2,1-b:3,4-b′]dithiophene-4-imine (CPDT=NR) polymers for use in organic solar cell applications and other electronic devices.

### BACKGROUND OF THE INVENTION

There is widespread interest in addressing global energy demand using renewable resources. See, e.g., “Solution-Processed Organic Solar Cells,” Brabec, C. J.; Durrant, J. R., MRS Bull. 2008, 33, 670-675, and “Polymer-Fullerene Bulk-Heterojunction Solar Cells,” Dennler, G.; Scharber, M. C.; Brabec, C. J., Adv. Mater. 2009, 21, 1323-1338. The earth absorbs more solar energy in one hour than the world uses in one year. Harnessing this vast amount of energy is a crucial scientific and socioeconomic challenge in view of the need for relieving carbon dioxide (CO₂) release into the environment and alleviating dependence on nonrenewable fossil fuels. Organic polymer devices are a third-generation solar technology that is rapidly emerging to compete with inorganic based first- and second-generation solar technologies and one possible solution attracting the attention of scientists, engineers, politicians and entrepreneurs as an auspicious source of alternative energy. The low cost synthesis of electrically tunable structures and inexpensive processing techniques capable of large-scale production onto lightweight flexible substrates makes polymer based solar cells a realizable technology in the near future. (See “Fabrication and Processing of Polymer Solar Cells: A Review of Printing and Coating Techniques,” Krebs, F. C., Sol. Energy Mater. Sol. Cells 2009, 93, 394-412; “Flexible Organic P3HT:PCBM Bulk-Heterojunction Modules With More than 1 Year Outdoor Lifetime,” Hauch, J. A.; Schilinsky, P.; Choulis, S. A.; Childers, R.; Biele, M.; Brabec, C. J., Sol. Energy Mater. Sol. Cells, 2008, 92, 727-731; and “Stability/Degradation of Polymer Solar Cells,” Jorgensen, M.; Norrman, K.; Krebs, F. C. Sol. Energy Mater. Sol. Cells, 2008, 92, 686-714.) One major obstacle facing polymer OPV technology is its low power conversion efficiency, which increases cost and limits applicability, as compared to inorganic counterparts (“Organic Photovoltaics,” Kippelen, B.; Bredas, J. L., Energy Environ. Sci. 2009, 2, 251-261 “Device Physics of Polymer: Fullerene Bulk Heterojunction Solar Cells,” Blom, P. W. M.; Mihailetchi, V. D.; Koster, L. A. J.; Markov, D. E., Device Physics of Adv. Mater. 2007, 19, 1551-1566; “‘Columnlike’ Structure of the Cross-Sectional Morphology of Bulk Heterojunction Materials,” Moon, J. S.; Lee, J. K.; Cho, S.; Byun, J.; Heeger, A. J., Nano Lett. 2009, 9, 230-234.)

There are four key steps in the conversion of sunlight to energy in a polymer OPV (organic photovoltaic) device: photo-excitation of the donor material (usually a conjugated polymer), by absorption of light to produce coulomb-correlated electron-hole pairs, i.e. excitons; diffusion of these excitons to the acceptor interface; dissociation of the excitons into charge carriers; and transport and collection of the separated charges. (See “Exciton Diffusion in Poly(p-phenylenevinylene)/C-60 Heterojunction Photovoltaic Cells,” Halls, J. J. M.; Pichler, K.; Friend, R. H.; Moratti, S. C., Holmes, Appl. Phys. Lett. 1996, 68, 3120-3122; “Photoinduced Carrier Generation in P3HT/PCBM Bulk Heterojunction Materials,” Hwang, I. W.; Moses, D.; Heeger, A. J., J. Phys. Chem. 2008, 112, 4350-4354; “Geminate Charge Recombination in Alternating Polyfluorene Copolymer/Fullerene Blends,” De, S.; Pascher, T.; Maiti, M.; Jespersen, K. G.; Kesti, T.; Zhang, F. L.; Inganas, O.; Yartsez, A.; Sundstrom, V., J. Am. Chem. Soc. 2007, 129, 8466-8472; “Photocurrent Generation in Polymer-Fullerene Bulk Heterojunctions,” Mihailetchi, V. D.; Koster, L. J. A.; Hummelen, J. C.; Blom, P. W. M., Phys. Rev. Lett. 2004, 93, 216601; “Spectroscopic Studies of Photoexcitation in Regioregular and Regiorandom Polythiophene Films,” R. A. J.; Vardeny, Z. V., Adv. Funct. Mater. 2002, 12, 587-597; “Why is Exciton Dissociation so Efficient at the Interface Between a Conjugated Polymer and an Electron Acceptor,” Arkhipov, V. I.; Heremans, P.; Bassler, H., Appl. Phys. Lett. 2003, 82, 4605-4607.) The most efficient devices employ a bulk heterojunction (BHJ) architecture, which utilizes an absorbing layer that consists of a blend of light-absorbing polymeric electron donor and a fullerene-based acceptor (“Polymer Photovoltaic Cells—Enhanced Efficiencies Via a Network of Internal Donor-Acceptor Heterojunctions,” Yu, G.; Gao, J.; Hummelen, J. C.; Wudl, F.; Heeger, A. J., Science 1995, 270, 1789-1791; “Plastic' Solar Cells: Self-Assembly of Bulk Heterojunction Nanomaterials by Spontaneous Phase Separation,” Peet, J.; Heeger, A. J.; Bazan, G. C., Acc. Chem. Res. 2009, 11, 1700-1708.) Power conversion efficiencies depend on several factors, including the donor-acceptor morphology, processing conditions, and orbital energy levels. (Organic Photovoltaics: Materials, Device Physics, and Manufacturing Technologies, Brabec, C. J.; Dyakonov, V., Wiley-VCH Verlag GmbH & Co. KGaA: Weinheim, Germany, 2008.) To optimize organic-based solar cells, extensive research efforts have aimed to design new polymer structures with optimized absorption overlap with the solar spectrum, high charge carrier mobilities, and optimized molecular orbital energy levels. Relatively high efficiency solar cells have been attained by annealing blends of regioregular poly(3-hexylthiophene) (rrP3HT) and [6,6]-phenyl-C₆₁-butyric acid methyl ester (C₆₁-PCBM). (See “Thermally Stable, Efficient Polymer Solar Cells With Nanoscale Control of the Interpenetrating Network Morphology,” Ma, W.; Yang, C.; Gong, X; Lee, K.; A. Heeger, A. J.; Adv. Funct. Mater. 2005, 15, 1617-1622.) Derivatives based on 4H-cyclopenta[2,1-b:3,4-b′]dithiophene (CPDT), where two thiophene units are fused and rigidified by a covalent carbon, have also attracted considerable interest. Due to the fully coplanar structure of CPDT, many intrinsic properties based on bithiophene can be altered, leading to extended conjugation, lower HOMO-LUMO energy band gaps and stronger intermolecular interactions. Polymers incorporating 4H-cyclopenta-[2,1-b:3,4-b′]-dithiophen-4-one (CPDT=O), as well as other derivatives and copolymers utilizing these precursors as comonomers, have attracted special interest for their electroactivity, n-type dopability and electrochromism (“Small Bandgap Polymers for Organic Solar Cells,” Kroon, R.; Lenes, M.; Hummelen, J. C.; Blom, P. W. M.; de Boer, B., Polym. Rev. 2008, 48, 531-582). These polymers and copolymers belong to a very promising group of conducting polymers with the lowest known band gaps in the range of 0.16 to 1.5 eV. Furthermore, D-A copolymers with a conjugated electron rich donor unit and a conjugated electron deficient acceptor unit incorporated into the polymer backbone introduce a push-pull driving force that facilitates electron delocalization and offers a powerful strategy in the design of low band gap conjugated polymers (see “Relationship Between Band-Gap and Bond Length Alternation in Organic Conjugated Polymers,” Bredas, J. L., J. Chem. Phys. 1985, 82, 3808-3811; “Design Rules for Donors in Bulk Heterojunction Solar Cells—Towards 10% Energy Conversion Efficiency,” Scharber M. C.; Mulbacher, D.; Koppe, M.; Denk, P.; Waldauf, C.; Heeger, A. J.; Brabec, C. J., Adv. Mater. 2006, 18, 789-794). Copolymers based on this architecture have led to high performance materials such as those based on poly[2,6-(4,4-bis(2-ethylhexyl)-4H-cyclopenta[2,1-b;3,4-b′]dithiophene)-alt-4,7-(2,1,3-benzothiadiazole)] (PCPDTBT) (“Panchromatic Conjugated Polymers Containing Alternating Donor/Acceptor Units for Photovoltaic Applications,” Zhu, Z.; Waller, D.; Gaudiana, R.; Morana, M.; Muhlbacher, D.; Scharber, M.; Brabec, C. J. Macromolecules 2007, 40, 1981-1986) which have achieved power conversion efficiencies (PCEs) of close to 6% (“Efficient Tandem Solar Cells Fabricated by All Solution Processing,” Kim, J. Y.; Lee, K.; Coates, N. E.; Moses, D.; Nguyen, T. Q.; Dante, M.; Heeger, A. J., Science 2007, 317, 222. 6306).

A universal architecture that allows for systematic derivitization via straightforward synthetic means would be of great utility in advancing the understanding of features related to materials performance in BHJ devices, and in preparing new and improved materials for use in such devices.

This invention describes one such architecture, that of novel imine-bridged CPDT=NR materials, and their synthesis, derivatives and applications in organic photovoltaic devices

### BRIEF SUMMARY OF THE INVENTION

The invention provides novel polymers, which are suitable for use in electronic devices such as solar cells, as well as novel monomer components of such polymers, and solar cell and other electronic devices incorporating such novel polymers.

In one embodiment, the invention provides a polymer of the formula (I):

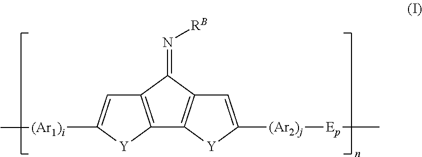

wherein R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl;

E_{P} is an electron-poor or electron-deficient aromatic moiety;
i is an integer independently selected from 0, 1, or 2;
j is an integer independently selected from 0, 1, or 2;
each Ar₁ and each Ar₂ are independently selected from unsubstituted C₆-C₂₀ aryl, substituted C₆-C₂₀ aryl, unsubstituted C₃-C₂₀ heteroaryl, and substituted C₃-C₂₀ heteroaryl;
n is an integer of at least about 5; and
Y is selected from the group consisting of S, —CH═CH—, Se, NH, NR¹ or Si, wherein R¹ is selected from C₁-C₂₄ hydrocarbyl.

In some embodiments, n is an integer of at least about 10. In some embodiments, n is an integer of at least about 20. In some embodiments, n is an integer of at least about 50. In some embodiments, n is an integer of at least about 100. In some embodiments, n is an integer between about 5 and about 10,000. In some embodiments, n is an integer between about 10 and about 10,000. In some embodiments, n is an integer between about 10 and about 5,000. In some embodiments, n is an integer between about 10 and about 2,500. In some embodiments, n is an integer between about 10 and about 1,000. In some embodiments, n is an integer between about 10 and about 500. In some embodiments, n is an integer between about 50 and about 10,000. In some embodiments, n is an integer between about 50 and about 5,000. In some embodiments, n is an integer between about 50 and about 2,500. In some embodiments, n is an integer between about 50 and about 1,000. In some embodiments, n is an integer between about 50 and about 500. In some embodiments, n is an integer between about 100 and about 10,000. In some embodiments, n is an integer between about 100 and about 5,000. In some embodiments, n is an integer between about 100 and about 2,500. In some embodiments, n is an integer between about 100 and about 1,000. In some embodiments, n is an integer between about 100 and about 500.

In some embodiments, the polymer can be terminated at its ends with a C₀-C₂₄ hydrocarbyl group, that is, it can be of the form:

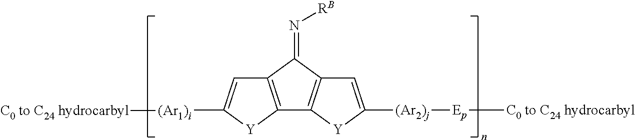

where R^{B}, E_{P}, I, j, Ar₁, Ar₂, Y, and n are as described herein.

In some embodiments, each Ar₁ and Ar₂ is independently selected from the group consisting of p-phenylene (para —C₆H₄—) and thiophene.

In some embodiments, each A₁ unit is identical to all other A₁ units. In some embodiments, each A₂ unit is identical to all other A₂ units. In some embodiments, each A₁ unit is identical to all other A₁ units, and each A₂ unit is identical to all other A₂ units. In some embodiments, each A₁ unit is identical to all other A₁ units, each A₂ unit is identical to all other A₂ units, and the A₁ unit and the A₂ are identical. In some embodiments, i is 1 and j is 1. In some embodiments, each A₁ unit is identical to all other A₁ units, i is 1, and j is 1. In some embodiments, each A₂ unit is identical to all other A₂ units, i is 1, and j is 1. In some embodiments, each A₁ unit is identical to all other A₁ units, each A₂ unit is identical to all other A₂ units, i is 1, and j is 1. In some embodiments, each A₁ unit is identical to all other A₁ units, each A₂ unit is identical to all other A₂ units, the A₁ unit and the A₂ are identical, i is 1, and j is 1. In some embodiments, i is 0 and j is 1. In some embodiments, each A₁ unit is identical to all other A₁ units, i is 0, and j is 1. In some embodiments, each A₂ unit is identical to all other A₂ units, i is 0, and j is 1. In some embodiments, each A₁ unit is identical to all other A₁ units, each A₂ unit is identical to all other A₂ units, i is 0, and j is 1. In some embodiments, each A₁ unit is identical to all other A₁ units, each A₂ unit is identical to all other A₂ units, the A₁ unit and the A₂ are identical, i is 0, and j is 1. In some embodiments, i is 1 and j is 0. In some embodiments, each A₁ unit is identical to all other A₁ units, i is 1, and j is 0. In some embodiments, each A₂ unit is identical to all other A₂ units, i is 1, and j is 0. In some embodiments, each A₁ unit is identical to all other A₁ units, each A₂ unit is identical to all other A₂ units, i is 1, and j is 0. In some embodiments, each A₁ unit is identical to all other A₁ units, each A₂ unit is identical to all other A₂ units, the A₁ unit and the A₂ are identical, i is 1, and j is 0.

In some embodiments, E_{P} is selected from substituted and unsubstituted moieties selected from the group consisting of thiadiazoloquinoxaline; quinoxaline; thienothiadiazole; thienopyridine; thienopyrazine; pyrazinoquinoxaline; benzothiadiazole; bis-benzothiadiazole; benzobisthiadiazole; thiazole; thiadiazolothienopyrazine; and diketopyrrolopyrrole. In some embodiments, E_{P} is selected from unsubstituted moieties selected from the group consisting of thiadiazoloquinoxaline; quinoxaline; thienothiadiazole; thienopyridine; thienopyrazine; pyrazinoquinoxaline; benzothiadiazole; bis-benzothiadiazole; benzobisthiadiazole; thiazole; thiadiazolothienopyrazine; and diketopyrrolopyrrole. In embodiments where E_{P} is substituted, E_{P} can be substituted with one or more C₁-C₂₄ hydrocarbyl groups or —O—C₁-C₂₄ hydrocarbyl groups. In some embodiments, E_{P} is selected from substituted and unsubstituted benzothiadiazole. In some embodiments, E_{P} is selected from

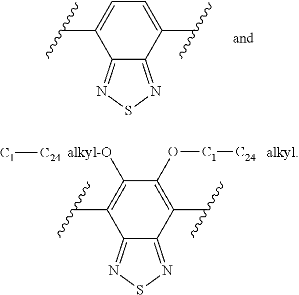

In one embodiment,

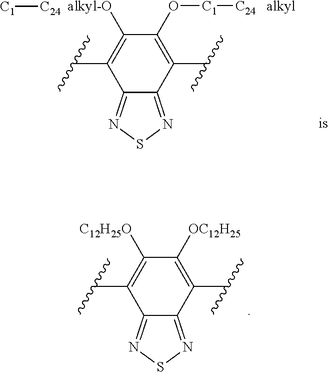

In any of the above embodiments, when the R^{B} moieties are substituted, they can be substituted with one or more substituents selected from the group consisting of F, Cl, Br, I, halogen, —R², —OH, —OR², —COOH, —COOR², —NH₂, —NHR², or NR²R³, where R² and R³ are independently selected from a C₁-C₂₄ hydrocarbyl group. In any of the above embodiments, R^{B} can be independently selected from

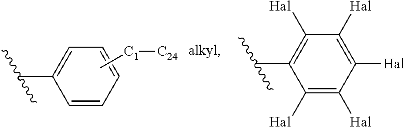

where Hal is halogen, or

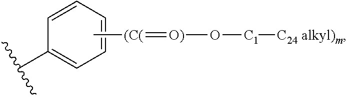

where m is 1 or 2. In one embodiment,

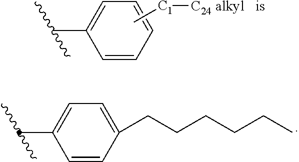

In one embodiment,

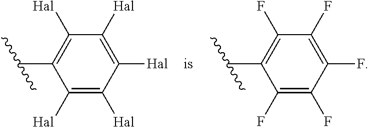

In one embodiment,

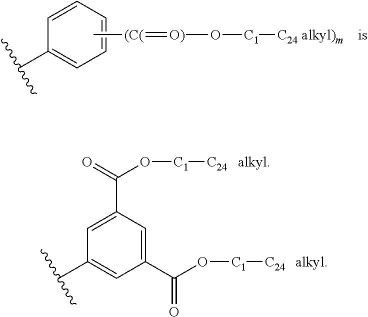

In one embodiment,

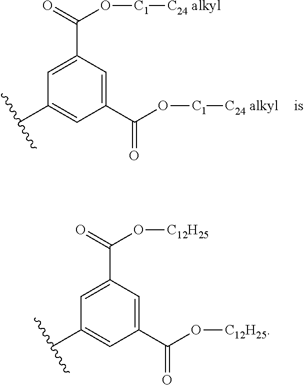

In any of the above embodiments, each R^{B} group on each monomer in the polymer can be selected independently. In any of the above embodiments, each R^{B} group on each monomer in the polymer is identical.

In any of the above embodiments, Y can be S. In any of the above embodiments, Y can be —CH═CH—.

In another embodiment, the invention provides a polymer of the formula (II):

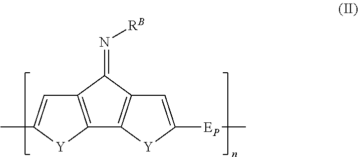

wherein R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl;

E_{P} is an electron-poor or electron-deficient aromatic moiety;
n is an integer of at least about 5; and
Y is selected from the group consisting of S, —CH═CH—, Se, NH, NR¹ or Si, wherein R¹ is selected from C₁-C₂₄ hydrocarbyl.

In some embodiments, n is an integer of at least about 10. In some embodiments, n is an integer of at least about 20. In some embodiments, n is an integer of at least about 50. In some embodiments, n is an integer of at least about 100. In some embodiments, n is an integer between about 5 and about 10,000. In some embodiments, n is an integer between about 10 and about 10,000. In some embodiments, n is an integer between about 10 and about 5,000. In some embodiments, n is an integer between about 10 and about 2,500. In some embodiments, n is an integer between about 10 and about 1,000. In some embodiments, n is an integer between about 10 and about 500. In some embodiments, n is an integer between about 50 and about 10,000. In some embodiments, n is an integer between about 50 and about 5,000. In some embodiments, n is an integer between about 50 and about 2,500. In some embodiments, n is an integer between about 50 and about 1,000. In some embodiments, n is an integer between about 50 and about 500. In some embodiments, n is an integer between about 100 and about 10,000. In some embodiments, n is an integer between about 100 and about 5,000. In some embodiments, n is an integer between about 100 and about 2,500. In some embodiments, n is an integer between about 100 and about 1,000. In some embodiments, n is an integer between about 100 and about 500.

In some embodiments, the polymer can be terminated at its ends with a C₀-C₂₄ hydrocarbyl group, that is, it can be of the form:

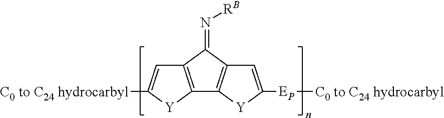

where R^{B}, E_{P}, Y, and n are as described herein.

In some embodiments, E_{P} is selected from substituted and unsubstituted moieties selected from the group consisting of thiadiazoloquinoxaline; quinoxaline; thienothiadiazole; thienopyridine; thienopyrazine; pyrazinoquinoxaline; benzothiadiazole; bis-benzothiadiazole; benzobisthiadiazole; thiazole; thiadiazolothienopyrazine; and diketopyrrolopyrrole. In some embodiments, E_{P} is selected from unsubstituted moieties selected from the group consisting of thiadiazoloquinoxaline; quinoxaline; thienothiadiazole; thienopyridine; thienopyrazine; pyrazinoquinoxaline; benzothiadiazole; bis-benzothiadiazole; benzobisthiadiazole; thiazole; thiadiazolothienopyrazine; and diketopyrrolopyrrole. In embodiments where E_{P} is substituted, E_{P} can be substituted with one or more C₁-C₂₄ hydrocarbyl groups or —O—C₁-C₂₄ hydrocarbyl groups. In some embodiments, E_{P} is selected from substituted and unsubstituted benzothiadiazole. In some embodiments, E_{P} is selected from

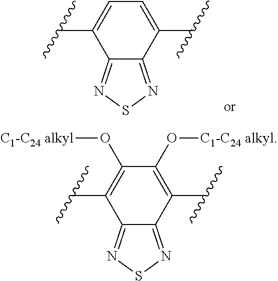

In one embodiment,

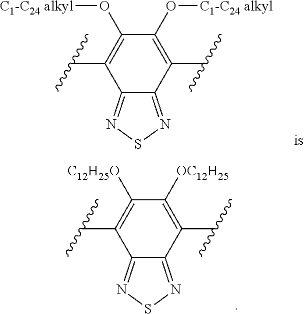

In any of the above embodiments, when the R^{B} moieties are substituted, they can be substituted with one or more substituents selected from the group consisting of F, Cl, Br, I, halogen, —R², —OH, —OR², —COOH, —COOR², —NH₂, —NHR², or NR²R³, where R² and R³ are independently selected from a C₁-C₂₄ hydrocarbyl group. In any of the above embodiments, R^{B} can be independently selected from

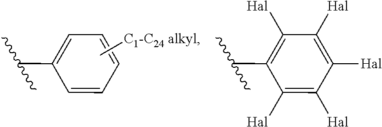

where Hal is halogen, or

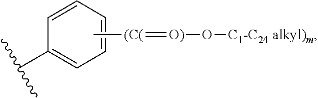

where m is 1 or 2. In one embodiment,

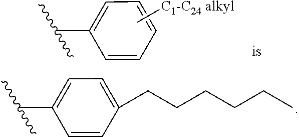

In one embodiment,

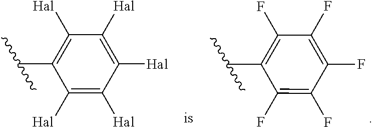

In one embodiment,

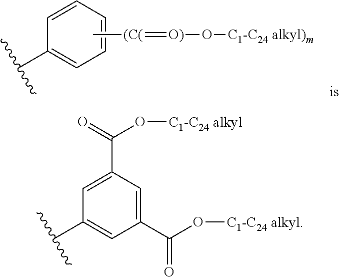

In one embodiment,

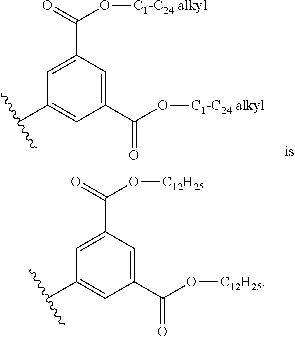

In any of the above embodiments, each R^{B} group on each monomer in the polymer can be selected independently. In any of the above embodiments, each R^{B} group on each monomer in the polymer is identical.

In any of the above embodiments, Y can be S. In any of the above embodiments, Y can be —CH═CH—.

In additional embodiments, the invention provides compounds of the formula (III):

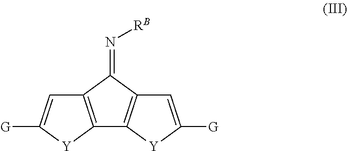

wherein: R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl; Y is selected from the group consisting of S, —CH═CH—, Se, NH, NR¹ or Si, wherein R¹ is selected from C₁-C₂₄ hydrocarbyl; and G is a leaving group.

Leaving group G can be a leaving group suitable for a Stille-type polymerization reaction, a leaving group suitable for a Suzuki-type polymerization reaction, or a leaving group suitable for a Yamamoto-type polymerization reaction. In some embodiments, G can be Br, Cl, I, triflate (trifluoromethanesulfonate), a trialkyl tin compound, boronic acid (—B(OH)₂), or a boronate ester (—B(OR₄)₂, where each R₄ is C₁-C₁₂ alkyl or the two R₄ groups combine to form a cyclic boronic ester of the form

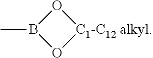

In some embodiments, G can be a trialkyl tin compound, such as (CH₃)₃—Sn—. In some embodiments, G can be

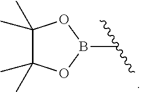 [SMILES: *B1OC(C)(C)C(C)(C)O1]

In some embodiments, G can be Br.

In any of the embodiments of Formula (III) above, Y can be S. In any of the embodiments of Formula (III) above, Y can be —CH═CH—.

In additional embodiments, the invention provides compounds of the formula (IV):

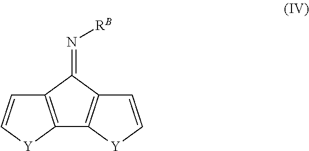

wherein R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl; and Y is selected from the group consisting of S, —CH═CH—, Se, NH, NR¹ or Si, wherein R¹ is selected from C₁-C₂₄ hydrocarbyl.

In further embodiments, the invention provides a device comprising:

a) a first hole-collecting electrode, optionally coated onto a transparent substrate;
b) an optional hole-transporting layer adjacent to the first electrode;
c) a bulk heterojunction layer (BHJ layer) comprising a polymer of Formula (I) or (II) and an electron acceptor;
d) an optional hole-blocking, exciton-blocking, or electron-transporting layer; and
e) a second electron-collecting electrode.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

### BRIEF DESCRIPTION OF THE DRAWINGS

FIG. 1 shows an ORTEP drawing of CPDT=NArBr₂, where Ar=2,6-diethyhlphenyl.

FIG. 2 shows the UV-Vis absorption spectra of P1 at 25° C. in o-dichlorobenzene (solid line) and cast as a thin film (dashed line).

FIG. 3 shows the UV-Vis absorption spectra of P2 at 25° C. in o-dichlorobenzene (solid line) and cast as a thin film (dashed line).

FIG. 4 shows the UV-Vis absorption spectra of P3 at 25° C. in o-dichlorobenzene (solid line) and the UV-Vis absorption spectra of P3 cast as a film from o-dichlorobenzene (dashed line).

FIG. 5 shows a current-voltage plot of a solar cell composed of P1:PC₇₁BM under air mass 1.5 global (AM 1.5G) irradiation at 100 MW cm⁻².

FIG. 6 shows the external quantum efficiency (EQE) spectra of the same device of FIG. 5.

FIG. 7 shows a current-voltage plot of solar cells composed of P1:PC₇₁BM under air mass 1.5 global (AM 1.5G) irradiation at 100 MW cm⁻² (dashed line). The device architecture for the solid line includes a MoO₃ interfacial layer and 2% DIO additive.

FIG. 8 shows the external quantum efficiency (EQE) spectra of the device employing a MoO₃ interfacial layer and 2% DIO additive.

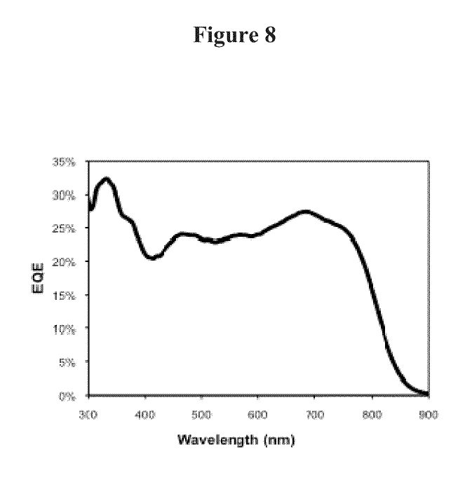

FIG. 9 shows a surface-phase image measured using atomic force microscopy (AFM) of the device employing a MoO₃ interfacial layer and 2% DIO additive.

### DETAILED DESCRIPTION OF THE INVENTION

### Definitions

“Alkyl” is intended to embrace a saturated linear, branched, cyclic, or a combination of linear and/or branched and/or cyclic hydrocarbon chain(s) and/or ring(s) having the number of carbon atoms specified, or if no number is specified, having 1 to 16 carbon atoms. “Alkenylene” is intended to embrace a divalent saturated linear, branched, cyclic, or a combination of linear and/or branched and/or cyclic hydrocarbon chain(s) and/or ring(s) having the number of carbon atoms specified, or if no number is specified, having 1 to 16 carbon atoms.

“Alkenyl” is intended to embrace a linear, branched, cyclic, or a combination of linear and/or branched and/or cyclic hydrocarbon chain(s) and/or ring(s) having at least one carbon-carbon double bond, and having the number of carbon atoms specified, or if no number is specified, having 2 to 16 carbon atoms. “Alkenylene” is intended to embrace a divalent linear, branched, cyclic, or a combination of linear and/or branched and/or cyclic hydrocarbon chain(s) and/or ring(s) having at least one carbon-carbon double bond, and having the number of carbon atoms specified, or if no number is specified, having 2 to 16 carbon atoms.

“Alkynyl” is intended to embrace a linear, branched, cyclic, or a combination of linear and/or branched and/or cyclic hydrocarbon chain(s) and/or ring(s) having at least one carbon-carbon triple bond, and having the number of carbon atoms specified, or if no number is specified, having 2 to 16 carbon atoms. “Alkynylene” is intended to embrace a linear, branched, cyclic, or a combination of linear and/or branched and/or cyclic hydrocarbon chain(s) and/or ring(s) having at least one carbon-carbon triple bond, and having the number of carbon atoms specified, or if no number is specified, having 2 to 16 carbon atoms.

“Hydrocarbyl” refers to an alkyl, an alkenyl, or an alkynyl group, or a combination of any or all of those groups, having the number of carbon atoms specified, or if no number is specified, having 1 to 16 carbon atoms (it will be appreciated by one of skill in the art that when a hydrocarbyl group is an alkenyl or alkynyl group, it must have at least two carbons, that is, C₂ to C₁₆; when a hydrocarbyl group has one carbon, it is necessarily an alkyl group, more specifically methyl). “Hydrocarbylene” refers to an alkylene, alkenylene, or alkynylene group, that is, a divalent group which is a combination of any or all of an alkylene, an alkenylene, or an alkynylene group having the number of carbon atoms specified, or if no number is specified, having 1 to 16 carbon atoms (it will be appreciated by one of skill in the art that when a hydrocarbylene group is an alkenylene or alkynylene group, it must have at least two carbons, that is, C₂ to C₁₆; when a hydrocarbylene group has one carbon, it is necessarily an alkylene group, more specifically methylene).

When a range such as “C₀-C₈ alkyl” or “C₀-C₃₂ hydrocarbyl” is used, the “C₀” value indicates that the moiety is a hydrogen. For example, in the substituent “—C₀-C₃₆ hydrocarbylene-C₆-C₂₀ aryl-C₀-C₃₆ hydrocarbyl,” when the C₀-C₃₆ hydrocarbyl group has the value of C₀, it is H (in this example, the substituent is then equivalent to —C₀-C₃₆ hydrocarbylene-C₆-C₂₀ aryl).

When a range such as “C₀-C₈ alkylene” or “C₀-C₃₂ hydrocarbylene” is used, the “C₀” value indicates that the moiety is absent. For example, in the substituent “—C₀-C₃₆ hydrocarbylene-C₆-C₂₀ aryl-C₀-C₃₆ hydrocarbyl,” when the —C₀-C₃₆ hydrocarbylene group has the value of C₀, it is absent (in this example, the substituent is then equivalent to —C₆-C₂₀ aryl-C₀-C₃₆ hydrocarbyl).

“Haloalkyl” indicates an alkyl group where at least one hydrogen of the alkyl group has been replaced with a halogen substituent, that is, a fluorine (F), chlorine (Cl), bromine (Br), or iodine (I) substituent. “Perhaloalkyl” indicates an alkyl group where all available valences have been substituted with halogen. For example, “perhaloethyl” can refer to —CCl₂CF₃, —CF₂CBr₃, or —CCl₂CCl₃.

“Fluoroalkyl” indicates an alkyl group where at least one hydrogen of the alkyl group has been replaced with a fluorine substituent. “Perfluoroalkyl” indicates an alkyl group where all available valences have been substituted with fluorine. For example, “perfluoroethyl” refers to —CF₂CF₃.

“Chloroalkyl” indicates an alkyl group where at least one hydrogen of the alkyl group has been replaced with a chlorine substituent. “Perchloroalkyl” indicates an alkyl group where all available valences have been substituted with chlorine. For example, “perchloroethyl” refers to —CF₂CF₃.

“Aryl” is defined as an optionally substituted aromatic ring system, such as phenyl or naphthyl. Aryl groups include monocyclic aromatic rings and polycyclic aromatic ring systems containing the number of carbon atoms specified, or if no number is specified, containing six to thirty carbon atoms. In other embodiments, aryl groups may contain six to twenty carbon atoms, six to twelve carbon atoms, or six to ten carbon atoms. In other embodiments, aryl groups can be unsubstituted. In other embodiments, aryl groups can be substituted. A preferred aryl group is phenyl.

“Heteroaryl” is defined as an optionally substituted aromatic ring system. Aryl groups contain the number of carbon atoms specified, and one or more heteroatoms (such as one to six heteroatoms, or one to three heteroatoms), where heteroatoms include, but are not limited to, oxygen, nitrogen, sulfur, and phosphorus. In other embodiments, aryl groups may contain six to twenty carbon atoms and one to four heteroatoms, six to twelve carbon atoms and one to three heteroatoms, or six to ten carbon atoms and one to three heteroatoms. In other embodiments, heteroaryl groups can be unsubstituted.

### Conjugated Polymers

The copolymers of the invention utilize a structure which permits an internal charge transfer (ICT) from an electron-rich unit to an electron deficient moiety. Conjugated polymers according to the invention are of the general form:

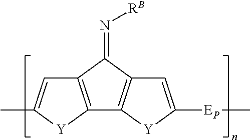

where R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl; E_{P} is an electron-poor aromatic moiety; n is an integer of at least about 5; and Y is selected from the group consisting of S, —CH═CH—, Se, NH, NR¹ or Si, where R¹ is selected from C₁-C₂₄ hydrocarbyl. In one preferred embodiment, Y is S. In another preferred embodiment, Y is —CH═CH—.

The size of the polymers can vary widely, depending on the properties desired, such as solubility, viscosity, etc. The number of repeat units, n, is an integer of at least about 5. In some embodiments, n is an integer of at least about 10, at least about 20, at least about 50, or at least about 100. In some embodiments, n is an integer between about 5 and about 10,000, between about 10 and about 10,000, between about 10 and about 5,000, between about 10 and about 2,500, between about 10 and about 1,000, between about 10 and about 500, between about 50 and about 10,000, between about 50 and about 5,000, between about 50 and about 2,500, between about 50 and about 1,000, between about 50 and about 500, between about 100 and about 10,000, between about 100 and about 5,000, between about 100 and about 2,500, between about 100 and about 1,000, or between about 100 and about 500. Other intervals, combining any of the above numerical parameters to form a new interval, can also be used (e.g., n between about 500 and 2,500).

The polymer can be terminated at its ends with a C₀-C₂₄ hydrocarbyl group, that is, it can be of the form:

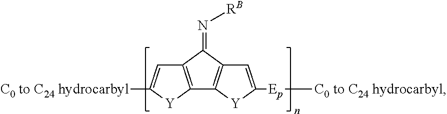

where the C₀-C₂₄ hydrocarbyl group can be, e.g., H, methyl, ethyl, etc.

### Synthesis of Conjugated Polymers

The synthesis of the polymers material starts from 4H-cyclopenta[2,1-b:3,4-b′]-dithiophen-4-one (CPDT=O) or similar materials (see Example 1 and other examples below; see “A New, Improved and Convenient Synthesis of 4H-cyclopenta[2,1-b:3,4-b′]-dithiophen-4-one,” Brzezinski, J. Z.; Reynolds, J. R., Synthesis 2002, 1053-1056). Starting materials which produce monomers suitable for polymerization, such as

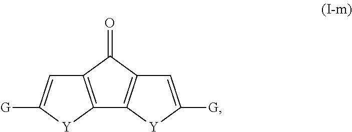

can be utilized, where G is a leaving group or a group suitable for a coupling reaction or a condensation reaction, such as Br, Cl, I, triflate (trifluoromethanesulfonate), boronic acid (—B(OH)₂), or a boronate ester (—B(OR₄)₂, where each R₄ is C₁-C₁₂ alkyl or the two R₄ groups combine to form a cyclic boronic ester of the form

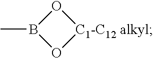

and Y is S (when G is Br and Y is S, this compound is then dibromo-4H-cyclopenta[2,1-b:3,4-b′]-dithiophen-4-one); Y can also be —CH═CH— (a fluorene-type structure), Se, Si, NH, or NR¹, where R¹ is a C₁-C₃₂ hydrocarbyl group). The leaving groups can be suitable for Suzuki coupling, Stille coupling, or Yamamoto coupling polymerization reactions to form polymers.

Treatment of CPDT=O or similar compounds with an amine of the form H₂NR^{B} and TiCl₄ affords compounds of the type CPDT=N—R^{B} or similar compounds:

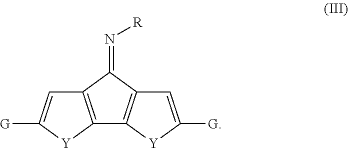

Compounds of Formula (III) can then be co-polymerized with other monomers in a Stille-type or Suzuki-type reaction, or in a Yamamoto-type reaction.

Polymers of Formula (I) are of the form of D-A conjugated polymers employing a CPDT=NR donor flanked by Ar units. The Ar units allow further alteration of the energy levels and fine-tuning of the electronic properties of the polymer. These polymers can be synthesized in an analogous manner using the appropriate acceptor unit (see Blouin, N.; Michaud, A.; Gendron, D.; Wakim, S.; Blair, E.; Neagu-Plesu, R.; Belletete, M.; Durocher, G.; Tao, Y.; Leclerc, M., J. Am. Chem. Soc. 2008, 130, 732-742) for synthetic details. Copolymerization of the bis-trimethylstannyl donor with a dibromo acceptor unit (i.e. Br—Ar₁-Eₚ-Ar₂—Br) will afford the desired copolymer. A variety of reagents and synthetic protocols widely utilized in the literature can be utilized to generate polymers of this architecture. (see “Synthesis of Conjugated Polymers for Organic Solar Cell Applications,” Cheng, Y. J.; Yang, S. H.; Hsu, C. S., Chem. Rev. 2009, 109, 5868-5923; see also Yokoyama et al., J. Am. Chem. Soc. 2007, 129, 7236-7237).

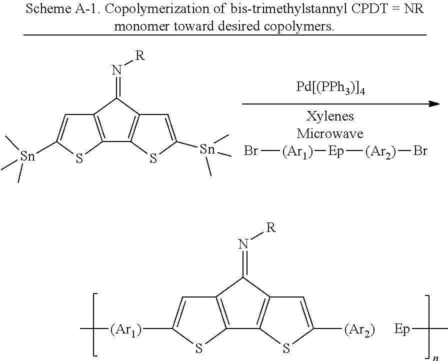

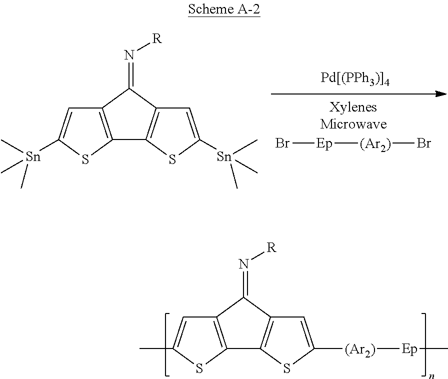

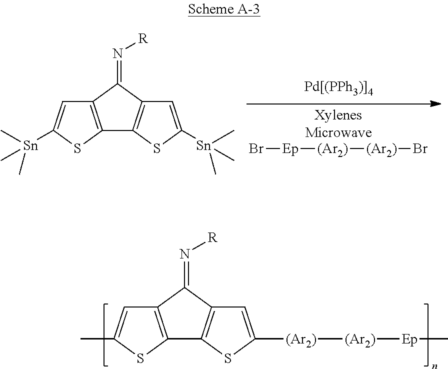

It should be noted that the dibromo reagent in Scheme A-1 can react in two different orientations (that is, Br—(Ar₁)-E_{P}-(Ar₂)—Br and Br—(Ar₂)-E_{P}-(Ar₁)—Br). The dibromo reagents in Scheme A-2 and Scheme A-3 can also react in two different orientations. When specific directional control of the addition of the (Ar₁)-E_{P}-(Ar₂) unit is desired, the reagents and synthesis depicted in either Scheme A-5 or Scheme A-6 can be employed.

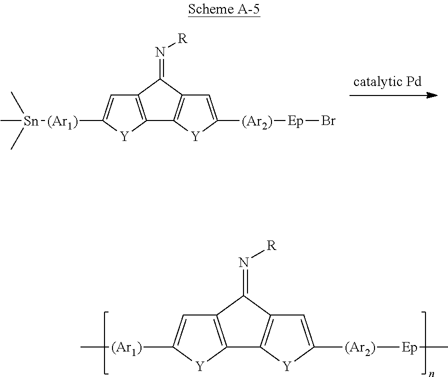

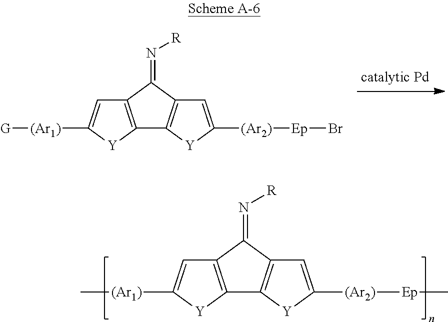

Additional illustrative syntheses of polymers of Formula (I) are shown in Schemes A-7 through A-10.

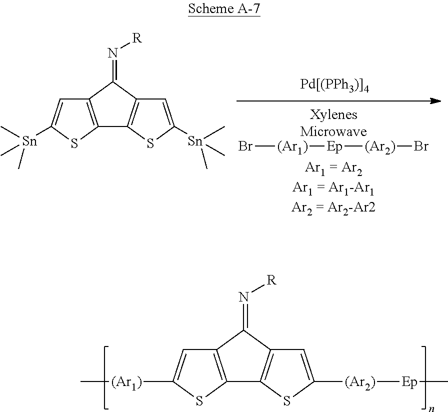

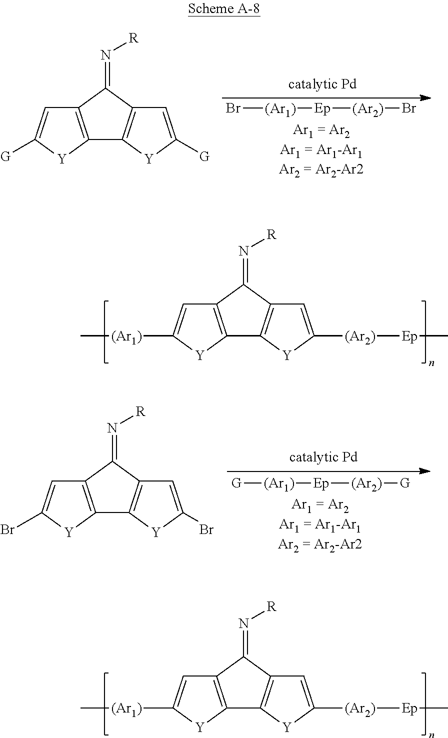

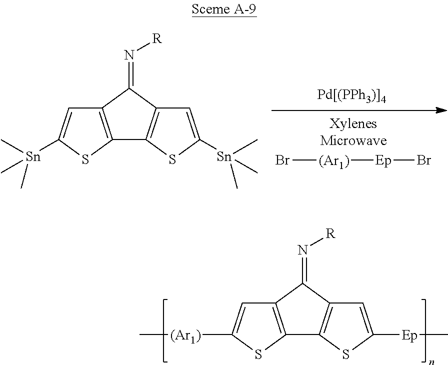

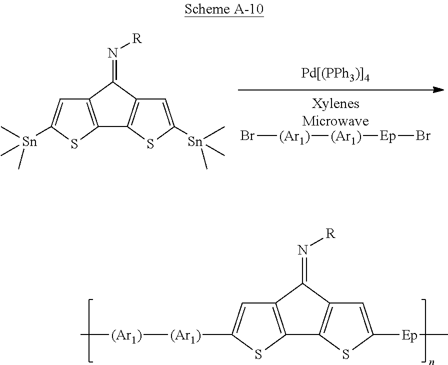

In the schemes above, G is a leaving group, such as a leaving group suitable for a Stille-type polymerization reaction, a Suzuki-type polymerization reaction, or a Yamamoto-type polymerization reaction.

### Electron-Poor Units

The “E_{P}” moieties which are used as intrachain units in the polymers can be any electron-deficient heteroaromatic ring system. “Electron-deficient aromatic ring system” and “electron-poor aromatic ring system” are used synonymously, and are intended to embrace 1) heteroaromatic ring systems, where the electron density on the carbon atoms of the heteroaromatic system is reduced compared to the analogous non-heteroaromatic system (see, for example, John A. Joule and Keith Mills, “Heterocyclic Chemistry, 5^{th} Edition” West Sussex, UK: Wiley, 2010, at page 7, Section 2.2.1.), and 2) aromatic ring systems, where the electron density on the carbon atoms of the aromatic system is reduced due to electron-withdrawing substituents on the aromatic ring (e.g., replacement of a hydrogen of a phenyl group with chlorine). While the E_{P} units in the polymer chain function as electron acceptors from the electron donor units in the polymer chain (for example, the electron-rich cyclopentadithiophenes), the E_{P} units are referred to as “electron-poor units” or “electron-deficient units” to avoid confusion with electron acceptors that do not form part of the polymer chain, such as fullerene-based electron acceptors or inorganic electron acceptors, that are used together with the polymers in an electronic device.

Examples of electron-poor intrachain units that can be used are:

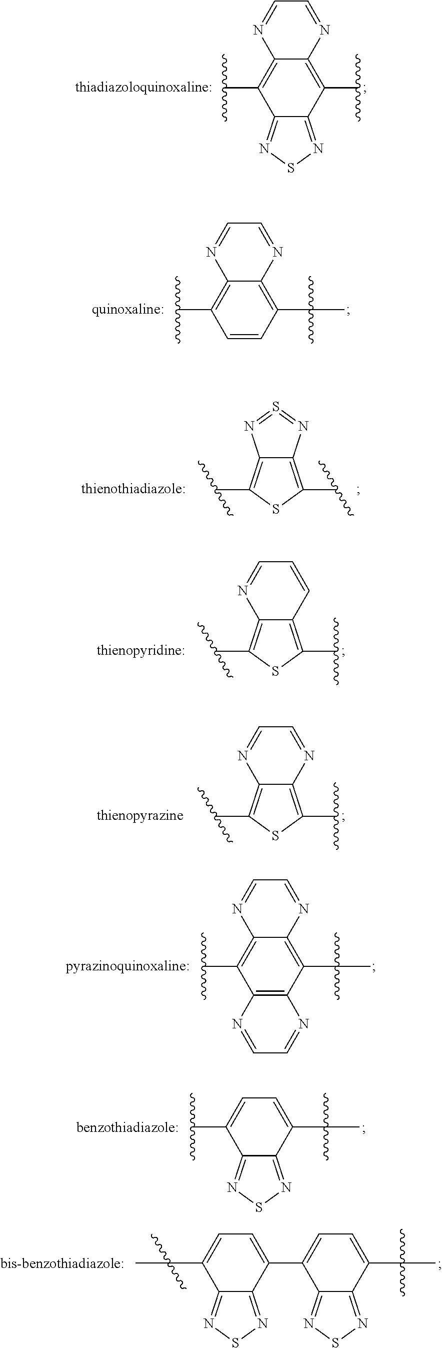

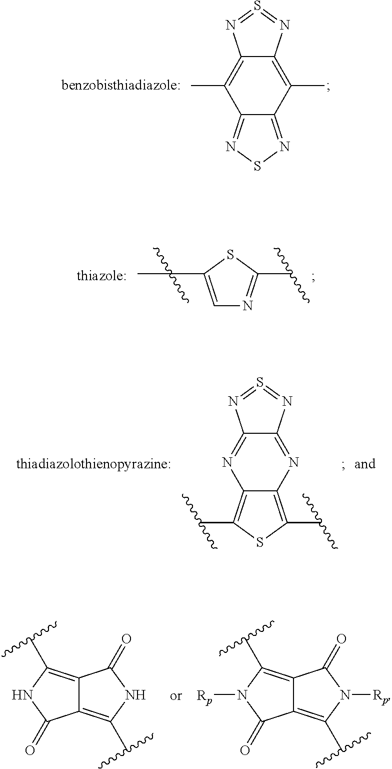

diketopyrrolopyrrole
where each Rₚ is independently C₁-C₈ alkyl, and preferably each Rₚ is the same moiety and is methyl or ethyl. In the structures above, the point of attachment of the E_{P} moiety to the remainder of the polymer is indicated as

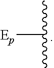

The E_{P} moieties can be unsubstituted, as depicted in the examples above (except the diketopyrrolopyrrole bearing the Rₚ groups, which is substituted with C₁-C₈ alkyl), or can be substituted. Substituents are added by replacing a hydrogen on the E_{P} moiety with the substituent moiety. Substituents include, but are not limited to, C₁-C₂₄ hydrocarbyl groups (that is, C₁-C₂₄ alkyl groups, C₂-C₂₄ alkenyl, C₂-C₂₄ alkynyl groups, or any combination of alkyl, alkenyl, and alkynyl fragments having between 1 and 24 carbon atoms), C₆-C₂₀ aryl groups (such as C₆-C₁₀ aryl groups, for example, phenyl), —O—C₁-C₂₄ hydrocarbyl groups (that is, —O—C₁-C₂₄ alkyl groups, —O—C₂-C₂₄ alkenyl groups, —O—C₂-C₂₄ alkynyl groups, or any combination of alkyl, alkenyl, and alkynyl fragments having between 1 and 24 carbon atoms attached to the remainder of the molecule via an oxygen atom), —OH, F, Cl, Br, I, and —C(═O)—O—C₁-C₂₄ hydrocarbyl groups (that is, —C(═O)—O—C₁-C₂₋₄ alkyl groups, —C(═O)—O—C₁-C₂₄ alkenyl groups, —C(═O)—O—C₁-C₂₄ alkynyl groups, or any combination of alkyl, alkenyl, and alkynyl fragments having between 1 and 24 carbon atoms attached to the remainder of the molecule via an —C(═O)—O— linkage). A preferred substituted E_{P} moiety is

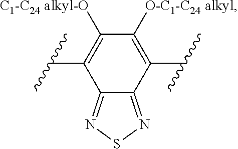

such as

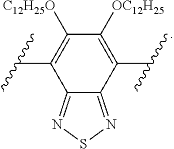

A preferred unsubstituted E_{P} moiety is

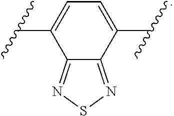 [SMILES: *c1ccc(*)c2nsnc12 ; "."]

### Imine Functionalization of CPDT, Fluorene, and Other Aromatic Structures

Introduction of an imine functionality at the bridgehead position of CPDT, fluorene, and other aromatic structures is a promising structural innovation that offers several advantages. The electron-deficient imine functionality can further reduce the HOMO-LUMO energies, the band gap, and is amenable to substitution with a wide range of backbone and pendant groups. This offers a unique opportunity to rapidly access a variety of steric and electronic variants, in an effort to better understand and control changes in the HOMO-LUMO energies, band-gap, polymer packing structure, and BHJ morphology, to understand the corresponding effects on the optoelectronic and charge transport properties of the polymers, and to generate novel materials (“Toward a Rational Design of Poly(2,7-carbazole) Derivatives for Solar Cells,” Blouin, N.; Michaud, A.; Gendron, D.; Wakin, S.; Blair, E.; Neagu-Plesu, R.; Belletete, M.; Durocher, G.; Tao, Y.; Leclerc, M., J. Am. Chem. Soc. 2008, 130, 732-742; “Charge Transport, Photovoltaic, and Thermoelectric Properties of Poly(2,7-carbazole) and Poly(indolo[3,2-b]carbazole) Derivatives,” Wakim, S.; Aich, B. R.; Tao, Y.; Leclerc, M., Polym. Rev. 2008, 48, 432-462; “Poly(2,7-carbazole)s: Structure-Property Relationships,” Blouin, N.; Leclerc, M., Acc. Chem. Res. 2008, 41, 1110-1119.) The rapid growth of the field has witnessed a wide variety of new polymer structures, only a few of which are high performing (see “Synthesis of Conjugated Polymers for Organic Solar Cell Applications,” Cheng, Y. J.; Yang, S. H.; Hsu, C. S., Chem. Rev. 2009, 109, 5868-5923). Functionalization at the bridging carbon allows greater structural variations for fine-tuning both the electronic and steric properties.

### Photovoltaic Devices Utilizing Polymers of the Invention

In one embodiment, the polymers of the invention are used in photovoltaic devices. In one embodiment, the device comprises the following layers:

a) a first hole-collecting electrode, optionally coated onto a transparent substrate;

b) an optional layer or layers adjacent to the first electrode, such as a hole-transporting layer;

c) a bulk heterojunction layer (BHJ layer) comprising a polymer of the invention and an electron acceptor;

d) an optional layer or layers such as hole-blocking, exciton-blocking, or electron-transporting layers; and

e) a second electron-collecting electrode.

It should be noted that the electron acceptor referred to in section c) is distinct from the electron-poor intrachain acceptor unit E_{P}. That is, the electron acceptor referred to in section c) is not part of the polymer chain itself, unlike the E_{P} unit, which is part of the polymer.

Typically, the first electrode can be transparent, allowing light to enter the device, but in some embodiments, the second electrode can be transparent. In some embodiments, both electrodes are transparent.

In another embodiment, the device comprises the following layers:

a′) indium tin oxide (ITO) coated glass (a first electrode);

b′) poly(3,4-ethylene dioxythiophene:poly(styrenesulfonate) (PEDOT:PSS);

c′) a bulk heterojunction layer (BHJ layer) comprising a polymer of the invention and an electron acceptor; and

d′) a metal electrode (a second electrode).

In one configuration, where light passes though a transparent first electrode (such as ITO-coated glass), it is absorbed by the BHJ layer, which results in the separation of electrical charges and migration of the charges to the electrodes, yielding a usable electrical potential.

The first electrode can be made of materials such as indium-tin oxide, indium-magnesium oxide, cadmium tin-oxide, tin oxide, aluminum- or indium-doped zinc oxide, gold, silver, nickel, palladium and platinum. Preferably the first electrode has a high work function (4.3 eV or higher).

In one embodiment, the first electrode, such as indium-tin oxide, has an optional interfacial layer of a hole-injecting material such as MoO₃, NiO, ReO₃, V₂O₅, WO₃, or RuOₓ, or another transition-metal oxide. The layer can be about 0.25 nm to about 10 nm thick, preferably about 0.5 nm to about 5 nm thick. This optional interfacial layer is located between the first electrode and the optional layer (b) in the device above, when layer (b) is present, or between the first electrode (a) and the bulk heterojunction layer (c) of the device above, or between the first electrode (a′) and PEDOT:PSS layer (b′) of the device above.

The optional layer adjacent to the first electrode is preferably polystyrenesulfonic acid-doped polyethylenedioxythiophene (PEDOT:PSS). Other hole transporting materials, such as polyaniline (with suitable dopants), or N,N′-diphenyl-N,N′-bis(3-methylphenyl)[1,1′-biphenyl]-4,4′-diamine (TPD), nickel oxide, can be used.

One method of fabricating the device is as follows: A conductive, transparent substrate is prepared from commercially available indium tin oxide-coated glass and polystyrenesulfonic acid-doped polyethylenedioxythiophene using standard procedures. A solution containing a mixture of the donor and acceptor materials is prepared so that the ratio of donor to acceptor is between 1:99 and 99:1 parts by mass; more preferably between 3:7 and 7:3 parts by mass. The overall concentration of the solution may range between 0.1 mg/mL and 100 mg/mL, but is preferably in the range of 10 mg/mL and 30 mg/mL.

The electron acceptor is preferably a fullerene, and more preferably [6,6]-phenyl C61-butyric acid methyl ester (PCBM), but may be a different fullerene (including, but not limited to, C71-PCBM), a tetracyanoquinodimethane, a vinazene, a perylene tetracarboxylic acid-dianhydride, a perylene tetracarboxylic acid-diimide, an oxadiazole, carbon nanotubes, or any other organic electron acceptor, such as those compounds disclosed in U.S. 2008/0315187.

In other embodiments, the electron acceptor is an inorganic acceptor selected from TiO₂ (titanium dioxide), TiOₓ (titanium suboxide, where x<2) and ZnO (zinc oxide). The titanium dioxide can be anatase, rutile, or amorphous. A titanium dioxide layer can be prepared by depositing a sol-gel precursor solution, for example by spincasting or doctorblading, and sintering at a temperature between about 300° C. and 500° C. When an inorganic material is used, the inorganic material can be dispersed in the polymer to create a single layer. Preparation of TiO₂ for use in solar cells is described in Brian O'Regan & Michael Gratzel, Nature 353:737 (1991) and Serap Giines et al., 2008 Nanotechnology 19 424009.

Useful solvents for preparing solutions of polymer or of polymer/acceptor, which solutions can then be deposited onto layers (a), (b), (d), or (e), or (a′), (b′), or (d′) of the devices above, include chloroform, toluene, chlorobenzene, methylene dichloride tetrahydrofuran, and carbon disulfide. However, the solvent used may be any solvent which dissolves or partially dissolve both donor and acceptor materials and has a non-zero vapor pressure.

The solution of polymer or of polymer/acceptor can be deposited onto the transparent conductive substrate by spin casting, doctor-blading, ink-jet printing, roll-to-roll coating or any process which yields a continuous film of the polymer or polymer-acceptor mixture, such that the thickness of the film is within the range of 10 to 1000 nm, more preferably between 50 and 150 nm.

In certain embodiments, the layer of the donor and acceptor is cast from a solution comprising a solvent and the electron donor and the electron acceptor. The solvent can comprise chloroform, thiophene, trichloroethylene, chlorobenzene, carbon disulfide, a mixture of any of the foregoing solvents or any solvent or solvent mixture that dissolves both the donor and acceptor organic small molecule. The solvent can also include processing additives, such as those disclosed in US Patent Application Publication Nos. 2009/0032808, 2008/0315187, or 2009/0108255. For example, 1,8-diiodooctane (DIO) can be added to the solvent/donor/acceptor mixture in an amount of 0.1-10% by volume. The additive, such as 2% DIO, can be added to any organic solvent used to cast the layer of donor/acceptor, such as chloroform. The solvent can also include doping agents such as molybdenum trioxide (MoO₃). For example, MoO₃ can be added to the solvent/donor/acceptor mixture in an amount of 0.1-10% by volume.

An additional layer or layers of material (i.e., the layer(s) adjacent to the second electrode) may optionally be deposited on the donor-acceptor film in order to block holes or excitons, act as an optical buffer, or otherwise benefit the electrical characteristics of the device. 2,9-dimethyl-4,7-diphenyl-1,10-phenanthroline can act as a hole-blocking or exciton-blocking material, while 4,4′,4″-tris[N-(3-methylphenyl)-N-phenylamino]triphenylamine and polyethylene dioxythiophene can act as exciton-blocking materials.

Finally, a metal electrode is deposited on top of the structure by thermal evaporation, sputtering, printing, lamination or some other process. The metal is preferably aluminum, silver or magnesium, but may be any metal such as gold, copper, platinum, or palladium, a conductive metal oxide, a conductive alloy, or a conductive polymer. In some embodiments, the device is annealed before and/or after deposition of the metal electrode.

### EXAMPLES

### Example 1

### Synthesis of N-4H-Cyclopenta-[2,1-b:3,4-b′]dithiophen-4-imine (CPDT=N) derivatives

The preparation of CPDT=O was carried out according to literature procedures (“A New, Improved and Convenient Synthesis of 4H-cyclopenta[2,1-b:3,4-b′]-dithiophen-4-one,” Brzezinski, J. Z.; Reynolds, J. R., Synthesis 2002, 1053-1056). Imine formation from the primary amine proceeds using typical condensation reaction procedures, but only for a limited number of substrates. This drastically limits the utility and synthetic variety that can be explored. Imines with any substantial bulk and certain functionalities cannot be generated utilizing this approach. It is well-established that the degree to which the polymers self-assemble into an optimally phase-separated BHJ morphology, and form ordered structures within the individual BHJ domains, is coupled to the intrinsic optical and electronic properties of the individual chains. Backbone substituents required for processing by solution methods can affect inter-chain packing and the BHJ interpenetrating network morphology. An improved synthetic strategy capable of rapidly accessing structurally diverse polymers was therefore developed for exploring structure-function relationships of both backbone and pendant groups, and for introducing substituents capable of electronic modulation.

Improved Synthesis of N-4H-Cyclopenta-[2,1-b:3,4-b′]dithiophen-4-imine (CPDT=N) derivatives

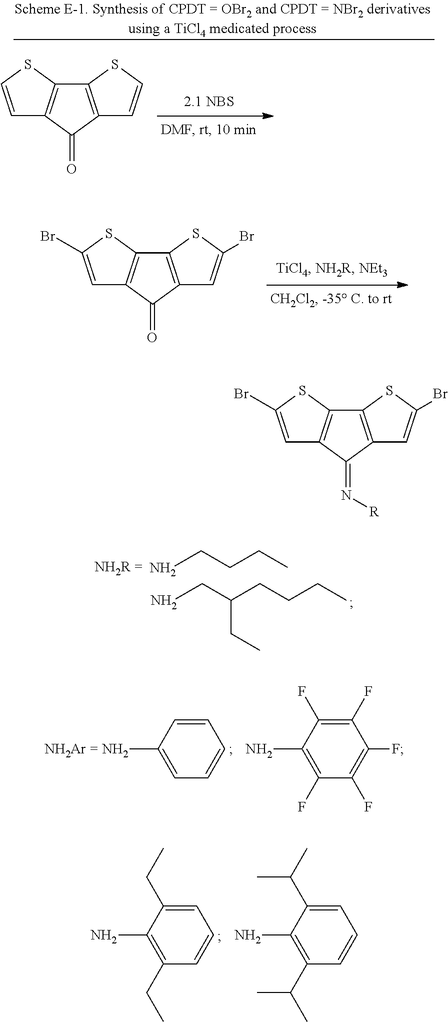

An improved synthetic route that allows for the straightforward synthesis of a library of derivatives from commercially available starting materials and is tolerant to a wide variety of functionality was developed. An improved method for the condensation of amines and anilines with CPDT=O was found which utilizes TiCl₄ and triethylamine in toluene and provides the desired monomers in greater than 90% conversion. In this reaction, a solution of TiCl₄ is added to a solution of amine or aniline and excess triethylamine at low temperature. A solution of dibromo-4H-cyclopenta-[2,1-b:3,4-b′]dithiophen-4-one (CPDT=OBr₂) is subsequently added and the reaction mixture is allowed to stir and warm to room temperature. Reaction times are substrate-dependent. The overall process is illustrated in Scheme E-1 utilizing derivatives for which typical condensation reactions failed. These derivatives were chosen on the basis that imine formation is sensitive to steric and electronic considerations. The addition of steric bulk at the ortho-positions of the aryl rings adds additional steric constraints, which demonstrates the utility of this methodology and illustrates that a wide variety of steric environments can be explored. Additionally, the successful installation of the pentafluorophenyl (C₆F₅) derivative illustrates that monomers with various substituents capable of modulating the electronics of the donor material can be accessed. This approach permits rapid generation of structures for evaluation of steric and electronic effects and allow for the introduction of various backbone substituents, solubilizing groups, and pendant groups. Additionally, the monomers can be generated rapidly, in almost quantitative yield directly from CPDT=OBr₂. The by-products of the reaction are triethylamine hydrochloride (HNEt₃Cl) and titanium dioxide (TiO₂), which can be removed by filtration; often no further purification will be required. This affords monomers that are ready to be polymerized by typical Suzuki routes.

### Example 2

### X-ray Crystal Structure of CPDT=N(2,6-Diethylphenyl)

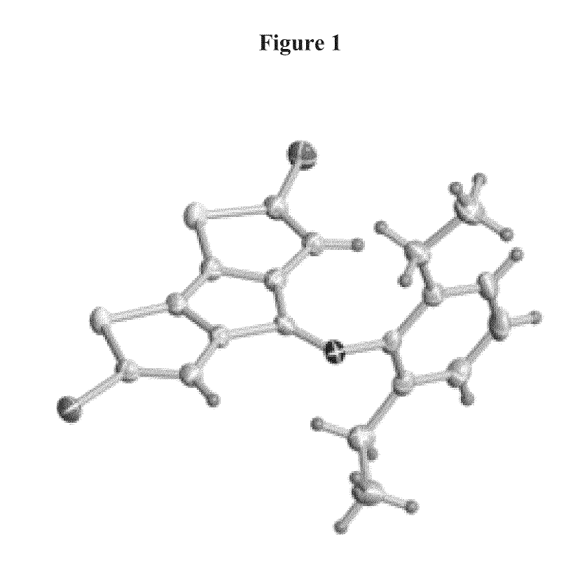

The X-Ray crystal structure of the 2,6-diethylphenyl derivative is shown in FIG. 1 and confirms the identity of the molecule. X-ray crystallography was performed as follows: a monocrystal was mounted on a glass fiber and transferred to a Bruker CCD platform diffractometer. The SMART program package (SMART Software Users Guide, Version 5.1, Bruker Analytical X-Ray Systems, Inc.; Madison, Wis. 1999) was used to determine the unit-cell parameters and for data collection (25 sec/frame scan time for a sphere of diffraction data). The raw frame data were processed using SAINT (SAINT Software Users Guide, Version 6.0, Bruker Analytical X-Ray Systems, Inc.; Madison, Wis. 1999) and SADABS (G. M. Sheldrick, SADABS, Version 2.05, Bruker Analytical X-Ray Systems, Inc.; Madison, Wis. 2001) to yield the reflection data file. Subsequent calculations were carried out using the SHELXTL program (G. M. Sheldrick, SHELXTL Version 6.12, Bruker Analytical X-Ray Systems, Inc.; Madison, Wis. 2001). The structure was solved by direct methods and refined on F² by full-matrix least-squares techniques. Analytical scattering factors for neutral atoms were used throughout the analysis (International Tables for X-Ray Crystallography 1992, Vol. C., Dordrecht: Kluwer Academic Publishers). Hydrogen atoms were located from a difference-Fourier map and refined (x,y,z and Uᵢₛₒ) (H. D. Flack, Acta. Cryst. 1983 A39, 876).

The imine functionality is in a planar arrangement with the bithiophene backbone, a unique environment compared to CPDT as the carbon at the bridgehead position is no longer tetrahedral. This structural feature can favor π-π stacking and allow for better inter-chain ordering. It is interesting to note that as the bulk at the ortho-positions on the aryl ring is increased (i.e. in going from H to 2,6-iPr₂), the aryl ring becomes more perpendicular to the bithiophene plane and the aryl ring becomes conformationally locked in the case of the bulkier (2,6-iPr₂)phenyl derivative as determined using NMR spectroscopy. This permits modification of inter-chain relationships in the bulk polymer based upon modification of the imine substituents on the monomer.

### Example 3

### Synthesis of Poly[(N-4H-Cyclopenta-[2,1-b:3,4-b′]dithiophen-4-imine)-alt-4,7-(2,1,3-benzothiadiazole)] D-A copolymers

D-A conjugated polymers used in high performance solar cells are readily synthesized using conventional Suzuki and Stille copolymerization reactions. These methodologies have been shown to be compatible with a wide variety of functionalities and are the main routes used for the desired copolymers. Suzuki reactions are the most widely utilized and can be carried out directly from the CPDT=NBr₂ precursors with commercially available 2,1,3-benzothiadiazole-4,7-bis(boronic acid pinacol ester) as illustrated in Scheme E-2, equation 1. In a typical Suzuki coupling reaction, a palladium catalyst such as Pd(PPh₃)₄ is used to carry out the polymerization in the presence of base (K₂CO₃), a phase-transfer catalyst and in a solvent mixture (typically toluene/water). (See “Synthesis, Characterization, and Photovoltaic Properties of a Low Band Gap Polymer Based on Silole-Containing Polythiophenes and 2,1,3-Benzothiadiazole,” Hou, J.; Chen, H. Y.; Zhang, S.; Li, G.; Yang, Y., J. Am. Chem. Soc. 2008, 130, 16144-16145.)

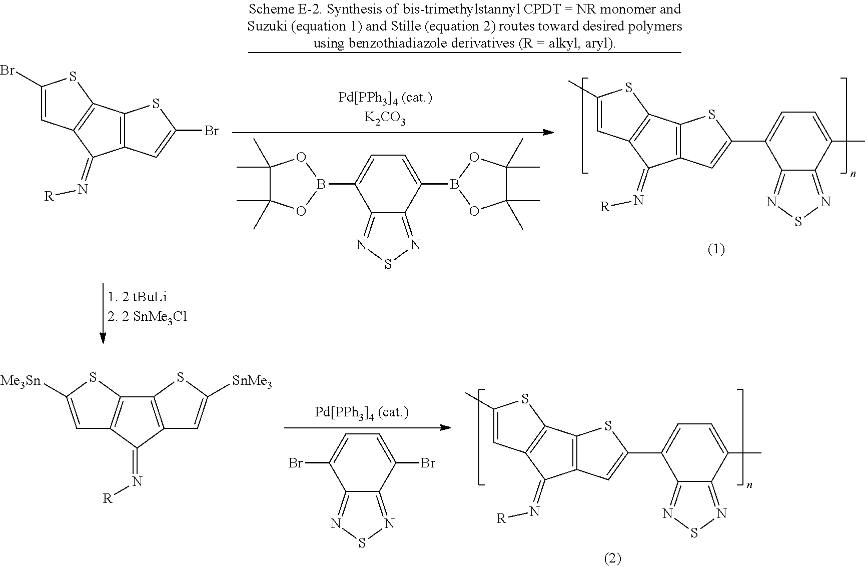

Scheme E-2, equation 2 schematically illustrates synthetic entry into the bis-trimethylstannyl CPDT=NR monomer and the subsequent Stille cross-coupling copolymerization with 4,7-dibromo-2,1,3-benzothiadiazole (BTBr₂) ((a) Microwave-Assisted Synthesis of Polythiophene Via the Stille Coupling, Tierney, S.; Heeney, M.; McCulloch, I.; Synth. Met. 2005, 148, 195-198; (b) Liquid-Crystalline Semiconducting Polymers With High Charge-Carrier Mobility, McCulloch, I.; Heeny, M.; Bailey, K.; Kristijonas, G.; MacDonald, I.; Shkunov, M.; Sparrowe, D.; Tierney, S.; Wagner, R.; Zhang, W. Chabinyc, M. L.; Kline, J. R.; McGehee, M. D.; Toney, M. F., Nat. Mater. 2006, 5, 328-333; (c) Bromination of 2,1,3-Benzothiadiazoles. Pilgram, K.; Zupan, M.; Skiles, R., J. Heterocyclic Chem. 1970, 7, 629-633). It is more reasonable that microwave assisted cross-coupling polymerizations will reproducibly afford copolymers with high number average molecular weights (Mₙ) in good yields, however, the two routes are compared and contrasted in the following Examples. A correlation between Mₙ and device performance has been demonstrated (Streamlined Microwave-Assisted Preparation of Narrow-Bandgap Conjugated Polymers for High Performance Bulk heterojunction Solar Cells, Coffin, R. C.; Peet, J.; Rogers, J.; Bazan, G. C.; Nat. Chem. 2009, 1, 657-661), and therefore it is important that a variety of methods are utilized in order to synthesize D-A copolymers of high Mₙ. The advantage of the Suzuki route is that no further synthetic manipulation of the monomer is required prior to polymerization.

### Example 4

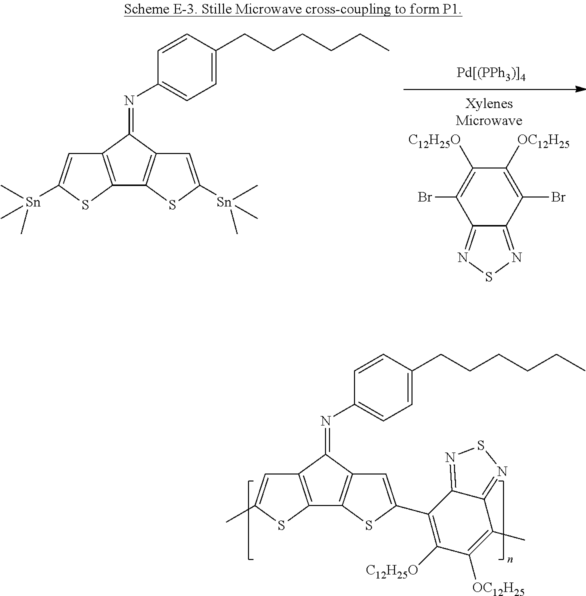

### Synthesis of Poly[(N-(2,6-cyclopenta[2,1-b:3,4-b′]dithiophen-4(7H)-ylidene)-4-hexylaniline-alt-5,6-bis(dodecyloxy)benzo[c][1,2,5]-thiadiazole] P1

The copolymerization of N-(2,6-bis(trimethylstannyl)cyclopenta[2,1-b:3,4-b′]-dithiophen-4(7H)-ylidene)-4-hexylaniline with 4,7-dibromo-5,6-bis(dodecyloxy)benzo-[c][1,2,5]-thiadiazole (BTOR₂Br₂) was carried out. The role of the hexyl and alkoxy substituents on the donor and acceptor, respectively, were to enhance solubility of the material so that precipitation or other unforeseen side effects did not hinder the polymerization reaction. Scheme E-3 shows the general polymerization scheme utilizing a microwave assisted Stille cross-coupling reaction. Microwave heating was targeted in lieu of conventional heating on the basis that many metal catalyzed reactions that generally require several hours to complete under conventional heating can be implemented in shorter times. (See “Ni(0)-Mediated Coupling Polymerization Via Microwave-Assisted Chemistry,” Carter, K; Macromolecules 2002, 35, 6757-6759; “Controlled Microwave Heating in Modern Organic Synthesis,” Kappe, C. O. Angew. Chem. Int. Ed. 2004, 43, 6250-6284; “Microwave-Assisted Preparation of Semiconducting Polymers,” Galbrect, F.; Bunnagel, T. W.; Scherf, U.; Farrell, T., Macromol. Rapid Commun., 2007, 29, 387-394.) By using this synthetic route and a 1.00:1.00 co-monomer molar ratio, it was possible to obtain a polymer in >80% yield with Mₙ=22 kg mo1⁻¹ and PDI=1.9 following purification via soxhlet extraction in a total reaction time of 50 minutes. The molecular weight of the material was determined by GPC at 150° C. in 1,2,4-trichlorobenzene relative to polystyrene standards.

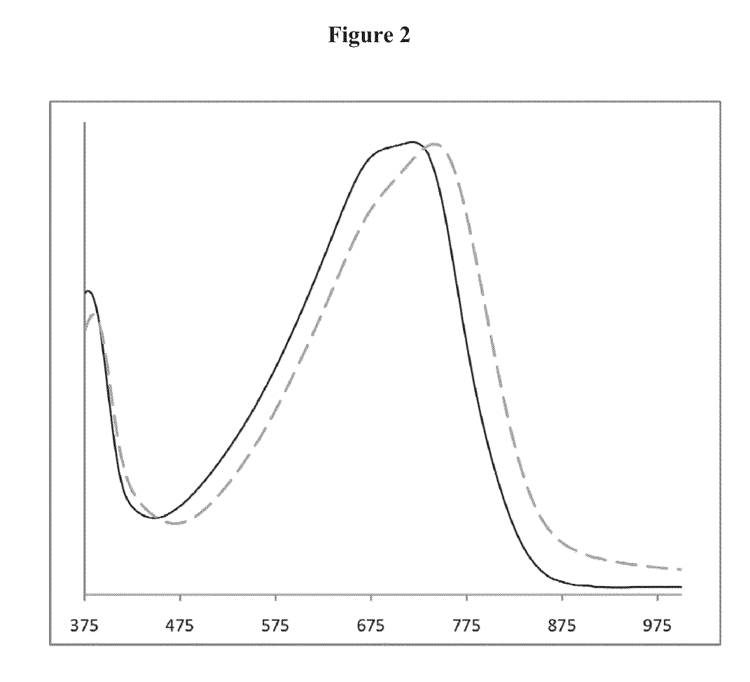

The UV-Vis absorption spectra of P1 at 25° C. in o-dichlorobenzene is shown in FIG. 2. The absorption maxima (λₘₐₓ) of P1 in solution occurs at 726 nm. The optical bandgap as estimated from the absorption onset of the film at 820 nm is 1.46 eV. The CV curves for the reduction and oxidation processes show that the HOMO is located at −5.38 eV and the LUMO was found at −3.73 eV, as determined by the oxidation and reduction onset, respectively. This gives an electrochemical bandgap of 1.65 eV. The HOMO-LUMO energies of P1 and PCPDTBT and electrochemical and optical bandgap are similar i.e. (1.51 and 1.40 eV respectively), which is regarded as ideal for polymer-fullerene BHJ solar cells. In agreement with computational efforts there is a lowering of the HOMO-LUMO energy levels of P1 relative to PCPDTBT. Photo-luminescence (PL) quenching studies with P1 illustrate that electron transfer to the fullerene takes place. When blended with PC₇₁BM, a BHJ device incorporating PCPDTBT achieves a PCE of up to 3.5%. Moreover, the photocurrent production is extended to wavelengths even longer than 900 nm. The high performance of PCPDTBT can be attributed to its broad, strong absorption spectrum and high mobility of charge carriers. The planar structure of PCPDTBT facilitates carrier transport between the polymer chains. It is reasonable to anticipate that polymers made using CPDT=NR derivatives will lead to a more planar structure, due to the planar arrangement of the bridgehead imine relative to the tetrahedral carbon in PCPDTBT. By incorporating a small amount of 1,8-octanedithiols into the PCPDTBT/PC₇₁BM solution prior to spin coating, the solar cell efficiency was further improved to 5.5% through the formation of an optimal BHJ morphology, which enhances both the photoconductivity and charge carrier lifetime. (See “Efficiency Enhancement in Low-Bandgap Polymer Solar Cells by Processing with Alkane Dithiols,” Peet, J.; Kim, J. Y.; Coates, N. E.; Ma, W. L.; Moses, D.; Heeger, A. J.; Bazan, G. C Nat. Mater. 2007, 6, 497-500; see also US 2009/0032808, US 2008/0315187, and US 2009/0108255).

P1 is as an excellent candidate for use in photovoltaic applications due to its excellent solubility in organic solvents, excellent film forming properties, high Mₙ, narrow PDI, optimal HOMO-LUMO energy levels, broad absorption characteristics, and appropriate absorption maximum (λₘₐₓ). The data here present a convenient route toward a new family of D-A conjugated polymers whose performance can be systematically investigated and tuned.

### N-(2,6-dibromocyclopenta[2,1-b:3,4-b′]-dithiophen-4(7H)-ylidene)-4-hexylandine

In a glovebox, a dry solution of 4-hexylaniline (535 mg, 3.02 mmol) and triethylamine (1.26 g, 12.48 mmol) in dichloromethane (10 ml) were chilled to −35° C. Titanium tetrachloride (541 mg, 2.85 mmol) in 1 mL toluene was added drop-wise over a period of 5 minutes to give a deep red solution, which was allowed to stir for an additional five minutes. A chilled solution of dibromo-4H-Cyclopenta-[2,1-b:3,4-b′]dithiophen-4-one (1.26 g, 3.02 mmol) was added at once. The solution was vigorously stirred, allowed to warm to room temperature and stirred overnight. Subsequently, 10 mL of diethyl ether was added and the resulting suspension was stirred over the course of an hour. The suspension was filtered through a silica plug and removal of the solvent gave 1.45 g (2.84 mmol) of the desired product (94.0%). ¹H NMR (500 MHz, [d₁]-chloroform, 298 K): δ=7.27 (s, 1H, Th—H), 7.21-7.19 (d, ³J_{HH}=8.0 Hz, 2H, ph-H), 6.88-6.87 (d, ³J_{HH}=8.0 Hz, 2 H, ph-H), 6.04 (s, 1H, Th—H), 2.66 (t, ³J_{HH}=7.65 Hz, 2H, ph-CH₂), 1.66 (quintet, ³J_{HH}=7.38 Hz, 2H, CH₂), 1.34 (m, 6H, CH₂), 0.90 (t, ³J_{HH}=6.6 Hz, 3H, CH₃), ¹³C NMR (125.7 MHz, [d₁]-chloroform, 298 K): 156.67 (imine), 154.80, 148.39, 145.70, 143.18, 142.89, 140.40, 135.04, 129.10, 126.68, 124.62, 119.90, 113.06, 111.57, 35.59, 31.87, 31.55, 29.00, 22.78, 14.26.

### N-(2,6-bis(trimethylstannyl)cyclopenta[2,1-b:3,4-b′]-dithiophen-4(7H)-ylidene)-4-hexylandine

In a glovebox, N-(2,6-dibromocyclopenta[2,1-b:3,4-b′]-dithiophen-4(7H)-ylidene)-4-hexylaniline (1.00 g, 1.96 mmol), Me₃SnSnMe₃ (1.93 g, 5.90 mmol) and Pd(PPh₃)₄ (116.0 mg, 0.100 mmol) were combined in a microwave tube and 2.5 mL of xylenes was added. The tube was sealed, removed from the glovebox and subjected to the following reaction conditions in a microwave reactor: 100° C. for 20 min, 120° C. for 20 min and 150° C. for 20 min. The mixture was poured into a separatory funnel containing 50 mL DI water and 50 mL of diethyl ether. The organic layer was further washed with 3×50 mL DI water. The organic layer was dried over anhydrous MgSO₄, and all volatiles were removed in vacuo. Purification by column chromatography on reverse phase silica using ethanol (containing 1% triethylamine) as the eluent gave 0.89 g (66.8%) of the product. ¹H NMR (500 MHz, [d₁]-chloroform, 298 K): δ=7.19 (s, 1H, Th—H), 7.12-7.10 (d, ³J_{HH}=8.1 Hz, 2H, ph-H), 6.86-6.85 (d, ³J_{HH}=8.1 Hz, 2H, ph-H), 5.86 (s, 1H, Th—H), 2.57 (t, ³J_{HH}=7.8 Hz, 2H, ph-CH₂), 1.57 (quintet, ³J_{HH}=7.6 Hz, 2H, CH₂), 1.26 (m, 6H, CH₂), 0.84 (t, ³J_{HH}=6.9 Hz, 3H, CH₃), 0.33 (s, 9H, Sn—CH₃), 0.17 (s, 9H, Sn—CH₃), ¹³C NMR (125.7 MHz, [d₁]-chloroform, 298 K): 156.67 (imine), 152.06, 149.66, 149.42, 147.69, 140.16, 139.96, 139.52, 138.30, 137.37, 137.30, 133.90, 133.77, 131.59, 129.19, 128.79, 128.74, 128.60, 128.56, 120.04, 35.65, 32.06, 31.88, 29.11, 22.74, 14.26, −8.19, −8.03.

### Poly[(N-(2,6-cyclopenta[2,1-b:3,4-b′]dithiophen-4(7H)-ylidene)-4-hexylaniline-alt-5,6-bis(dodecyloxy)benzo[c][1,2,5]-thiadiazole] P1

A microwave tube was charged with 100.0 mg (0.151 mmol) of 4,7-dibromo-5,6-bis(dodecyloxy)benzo-[c][1,2,5]-thiadiazole, and N-(2,6-bis(trimethylstannyl)cyclopenta[2,1-b:3,4-b′]-dithiophen-4(7H)-ylidene)-4-hexylaniline (102.2 mg, 0.151 mmol, 1.00 equivalents). The tube was brought inside a glovebox and 3-4 mg of Pd(PPh₃)₄ and 1 ml of xylenes was added. The tube was sealed, removed from the glovebox and subjected to the following reaction conditions in a microwave reactor: 120° C. for 5 min, 140° C. for 5 min and 170° C. for 40 min. After this time the reaction was allowed to cool leaving a viscous liquid containing some solid material. The mixture was dissolved in hot 1,2-dichlorobenzene, then precipitated into methanol and collected via centrifugation. The residual solid was loaded into an extraction thimble and washed successively with methanol (4 h), acetone (4 h), hexanes (12 h), and again with acetone (2 h). The polymer was dried in-vacuo to give 105 mg (0.123 mmol, 81.4%) of a blue solid. Analysis via GPC at 150° C. in 1,2,4-trichlorobenzene relative to polystyrene standards led to P1 with Mₙ of 22 kg mol⁻¹ and PDI=1.9. ¹H NMR (500 MHz, [d₄]-1,2-dichlorobenzene, 385 K): δ=8.84 (br m, 1H, Th—H), δ=7.92 (br m, 1H, Th—H), 7.31 (br m, 4H, ph-H, 4.18), (br m, 4H, OCH₂), 2.74 (br m, 2H, ph-CH₂), 2.06-1.94 (br, 4H, CH₂), 1.77 (br m, 2H, CH₂), 1.23 (br m, 42H, CH₂), 0.80 (br m, 9H, CH₃).

### Example 5

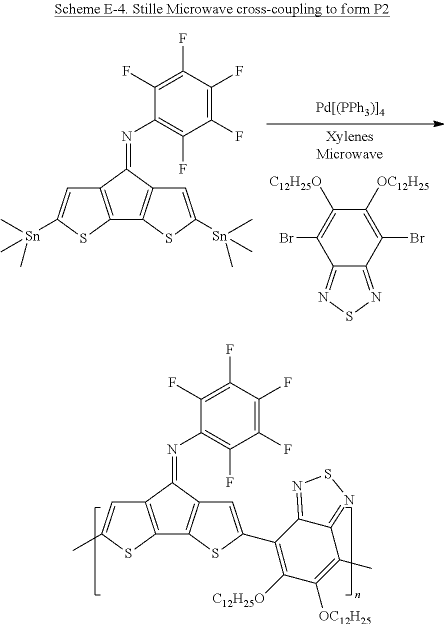

### Poly[(N-(2,6-cyclopenta[2,1-b:3,4-b′]dithiophen-4(7H)-ylidene)-4-pentafluoroaniline-alt-5,6-bis(dodecyloxy)benzo-[c][1,2,5]-thiadiazole] P2

To examine the effect of how various substituents can be utilized to modify the HOMO-LUMO energies, band-gap, and optical and optoelectronic properties, the synthesis of P2, bearing an electron withdrawing pentafluorophenyl substituent was carried out. N-(2,6-bis(trimethylstannyl)cyclopenta[2,1-b:3,4-b]-dithiophen-4(7H)-ylidene)-pentafluoroaniline was prepared in an analogous procedure as described in Example 3. A microwave tube was charged with 96.0 mg (0.146 mmol) of 4,7-dibromo-5,6-bis(dodecyloxy)benzo-[c][1,2,5]-thiadiazole, and N-(2,6-bis(trimethylstannyl)-cyclopenta[2,1-b:3,4-b′]-dithiophen-4(7H)-ylidene)-pentafluoroaniline 100.0 mg (0.146 mmol, 1.00 equivalents). The tube was brought inside a glovebox and 3-4 mg of Pd(PPh₃)₄ and 1 ml of xylenes was added. The tube was sealed, removed from the glovebox and subjected to the following reaction conditions in a microwave reactor: 120° C. for 5 min, 140° C. for 5 min and 180° C. for 40 min After this time the reaction was allowed to cool, leaving a viscous liquid containing some solid material. The mixture was dissolved in hot 1,2-dichlorobenzene, then precipitated into methanol and collected via centrifugation. The residual solid was loaded into an extraction thimble and washed successively with methanol (4 h), acetone (4 h), hexanes (12 h), and again with acetone (2 h). The polymer was dried in-vacuo to give 98 mg (0.114 mmol, 78.0%) of a blue solid. Analysis via GPC at 150° C. in 1,2,4-trichlorobenzene relative to polystyrene standards led to P2 with Mₙ of 44 kg mol⁻¹ and PDI=2.5. ¹H NMR (500 MHz, [d₁]-chloroform, 315 K): δ=8.80 (br m, 1H, Th—H), 7.87 (br m, 1H, Th—H), 4.18 (br, 4H, OCH₂), 1.95 (br, 4H, CH₂), 1.28 (br m, 36H, CH₂), 0.84 (br m, 6H, CH₃), ¹⁹F NMR (470 MHz, [d₁]-chloroform, 315 K): δ −150.86, −161.45, −162.57.

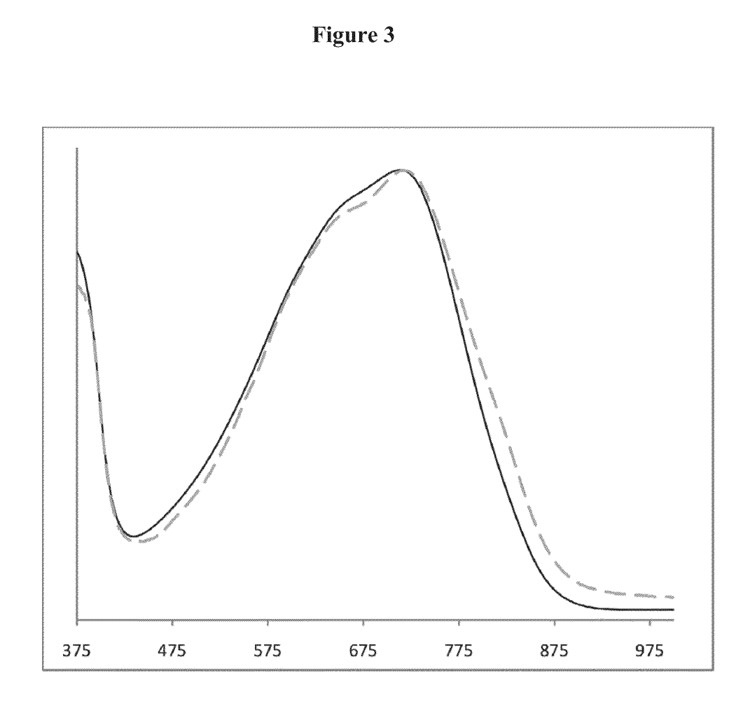

The UV-Vis absorption spectra of P2 at 25° C. in o-dichlorobenzene is shown in FIG. 3. The absorption maxima (λₘₐₓ) of P1 in solution occurs at 717 nm. The optical bandgap as estimated from the absorption onset of the film at 878 nm is 1.41 eV. The CV curves for the reduction and oxidation processes show that the HOMO is located at −5.61 eV and the LUMO was found at −4.12 eV, as determined by the oxidation and reduction onset, respectively. This gives an electrochemical bandgap of 1.49 eV.

### Example 6

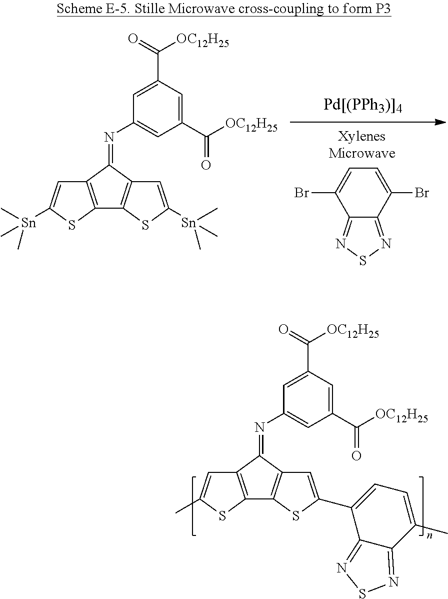

### Poly[(didodecyl-5-(2-cyclopenta[2,1-b:3,4-b′]dithiophen-4(7H)-ylideneamino)isophthalate-alt-4,7-(2,1,3-benzothiadiazole)] P3

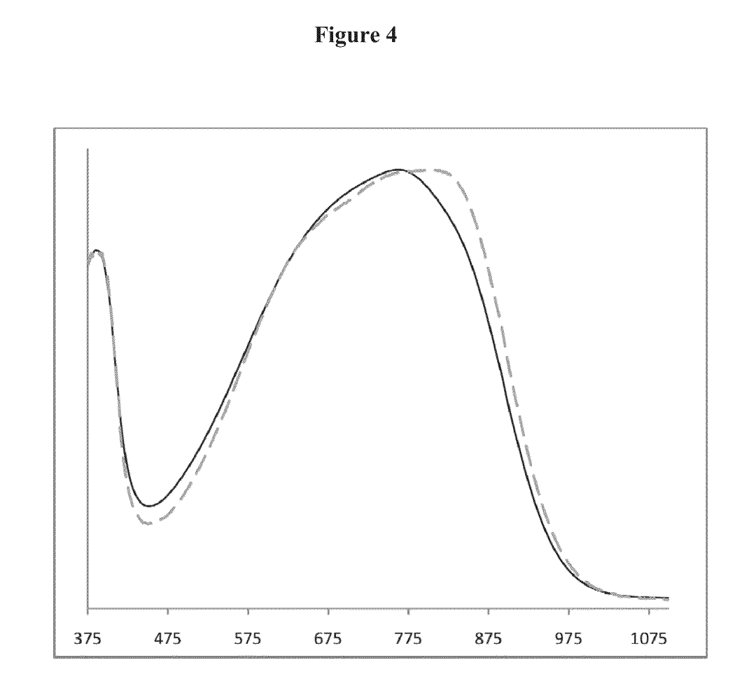

Didodecyl-5-(2,6-bis(trimethylstannyl)cyclopenta[2,1-b:3,4-b′]dithiophen-4(7H)-ylideneamino)isophthalate was prepared in an analogous procedure as described in Example 3. A microwave tube was charged with 43.0 mg (0.147 mmol) of 4,7-(2,1,3-benzothiadiazole), and didodecyl-5-(2,6-bis(trimethylstannyl)cyclopenta[2,1-b:3,4-b′]dithiophen-4(7H)-ylideneamino)isophthalate 150.0 mg (0.147 mmol, 1.00 equivalents). The tube was brought inside a glovebox and 3-4 mg of Pd(PPh₃)₄ and 1 ml of xylenes was added. The tube was sealed, removed from the glovebox and subjected to the following reaction conditions in a microwave reactor: 120° C. for 5 min, 140° C. for 5 min and 170° C. for 40 min. After this time the reaction was allowed to cool leaving a viscous liquid containing some solid material. The mixture was dissolved in hot 1,2-dichlorobenzene, then precipitated into methanol and collected via centrifugation. The residual solid was loaded into an extraction thimble and washed successively with methanol (4 h), acetone (4 h), THF (12 h), and again with acetone (2 h). The polymer was dried in-vacuo to give 81 mg (0.098 mmol, 66.6%) of a blue solid. Analysis via GPC at 150° C. in 1,2,4-trichlorobenzene relative to polystyrene standards led to P3 with Mₙ of 23 kg mol⁻¹ and PDI=1.8. ¹H NMR (500 MHz, [d₁]-chloroform, 315 K): δ=8.69-7.97 (br m, 7H), 4.43 (br m, 4H), 1.52-0.89 (br m, 46H). Heating the sample to 385 K in [d₄]-oDCB did not result in a more resolved NMR spectra, probably as a result of aggregation of the polymer. The UV-Vis absorption spectra of P3 at 25° C. in o-dichlorobenzene is shown in FIG. 4. The absorption maxima (λₘₐₓ) of P1 in solution occurs at 783 nm. The optical bandgap as estimated from the absorption onset of the film at 970 nm is 1.28 eV. The CV curves for the reduction and oxidation processes show that the HOMO is located at −5.26 eV and the LUMO was found at −4.16 eV, as determined by the oxidation and reduction onset, respectively. This gives an electrochemical bandgap of 1.1 eV.

Electrochemistry.

Electrochemical characteristics were determined by cyclic voltammetry (50 mV/s) carried out on drop-cast polymer films at room temperature in degassed anhydrous acetonitrile with tetrabutylammonium hexafluorophosphate (0.1 M) as the supporting electrolyte. The working electrode was glassy carbon, the counter electrode was a platinum wire, and the reference electrode (RE) was a silver wire. After each measurement the RE was calibrated with ferrocene (oxidation potential E₀=400 mV versus normal hydrogen electrode (NHE)) and the potential axis was corrected to NHE (using −4.75 eV for NHE) according to the difference between E₀ (ferrocene) and the measured E_{1/2} (ferrocene). HOMO and LUMO levels were estimated from oxidation and reduction onsets.

### Example 7

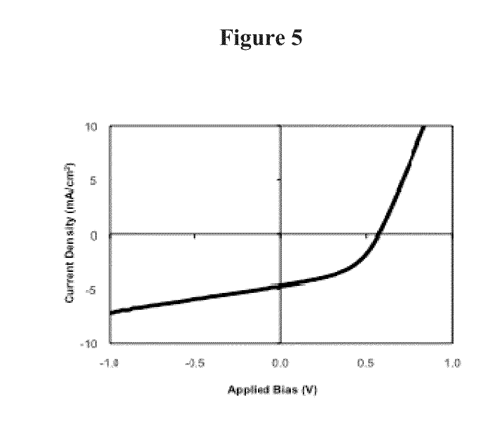

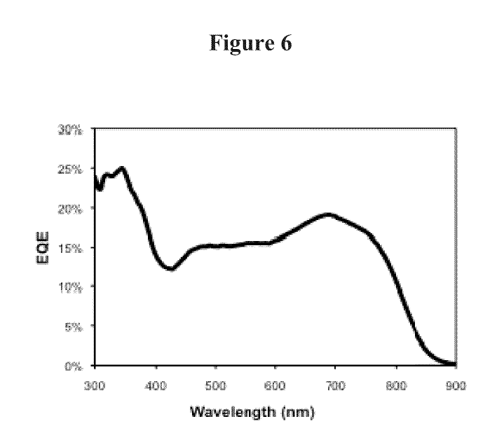

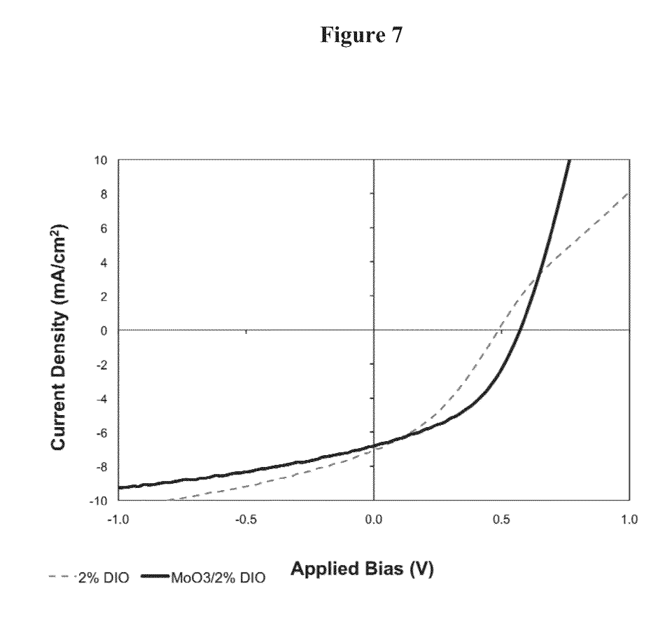

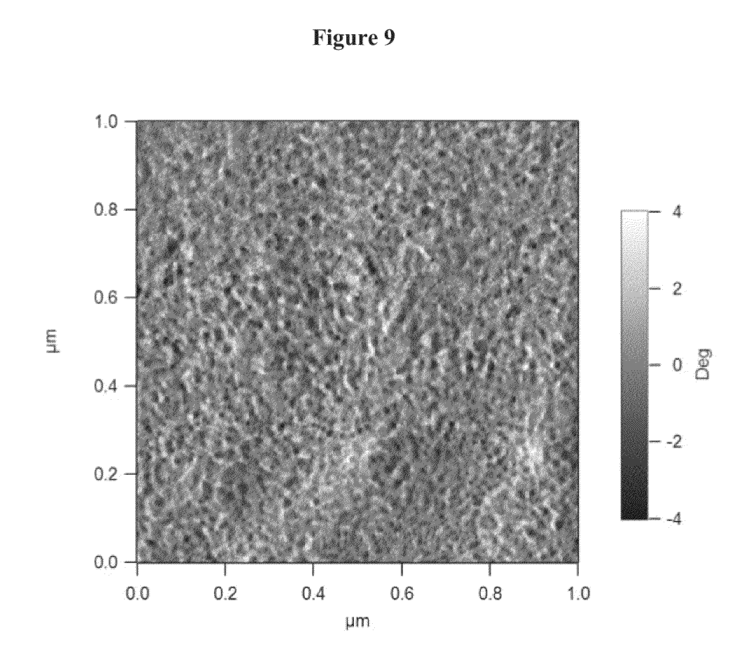

Use of P1 in bulk heterojunction organic photovoltaic devices is discussed. Device fabrication was carried out using standardized methodologies and previously published procedures as detailed below. The PEDOT:PSS (polyethylene dioxythiophene doped with polystyrene sulfonate) hole injection layer and polymer/PC₇₁BM, were spin-cast onto an indium-doped tin oxide (ITO) coated glass substrate, followed by deposition of the aluminum cathode. Performance optimization i.e. varying the polymer/fullerene ratio, spin speeds, polymer concentration in solution, solvent and polymer processing additive is expected to lead to improved performance. FIG. 5 and FIG. 6 illustrate the current-voltage characteristics of a solar cell composed of P1:PC₇₁BM under air mass 1.5 global (AM 1.5G) irradiation at 100 MW cm⁻² and the external quantum efficiency (EQE) spectra of the same device using P1. The J-V characteristics of the device for P1 with a 1:2 fullerene ratio show that the short circuit current density (J_{SC}) of the device is −4.75 with an open circuit voltage (V_{oc}) of 0.58 and a fill factor of 0.46. The EQE illustrates that current generation extends to approximately 900 nm, which lead to a power conversion efficiency ( ₑ) of 1.26%. FIG. 7 illustrates the use of an MoO₃ interfacial layer and diiodooctane (DIO) additive for improving both the performance and stability of the device. The J-V characteristics of the device for P1 with a 1:2 fullerene ratio show that the short circuit current density (J_{SC}) of the device is −6.82 with an open circuit voltage (V_{oc}) of 0.58 and a fill factor of 0.43. The EQE illustrates that current generation extends to approximately 900 nm, which lead to a power conversion efficiency ( ₑ) of 1.70%. FIG. 9 illustrates that defined nanoscale phase separation is observed in the surface phase images measured by atomic force microscopy (AFM).

These data illustrate that D-A copolymers with imine substituents at the donor bridgehead position can be used as the donor material in bulk heterojunction solar cells with PCBM acceptors.

### Device Fabrication and Characterization.

Indium tin oxide (ITO, 140 nm)-coated AMLCD glass substrates (purchased from Thin Film Devices) were sonicated for 10 min in isopropanol and treated in a UV ozone cleaner for an hour. Poly(3,4-ethylene dioxythiophene/poly(styrene-sulfonate) (PEDOT:PSS, Baytron P) was cast onto the prepared substrates by spin casting at 5000 rpm for 60 seconds then annealing at 140° C. for 30 minutes yielding a conductive PEDOT:PSS film of ˜50 nm. A blend solution of the copolymers and PC₇₁BM (Nano-C, USA) in chlorobenzene was formulated and filtered through a 1.0 μm Whatman poly(tetrafluoroethylene) (PTFE) syringe filter prior to spin coating on top of the PEDOT:PSS layer. 17 mm² aluminum electrodes (100 nm) were subsequently thermally evaporated at 1×10⁻⁷ torr using a shadow mask. The best P1 devices tested used a solution comprising 10 mg/mL of P1 and 20 mg/mL of PC₇₁BM spun cast at 1500 rpm from chlorobenzene. The best MoO₃ devices tested used a solution comprising 10 mg/mL of P1 and 10 mg/mL of PC₇₁BM spun cast at 800 rpm from chlorobenzene containing 2% diiodooctane. Solar cells were characterized under simulated 100 MW cm⁻² AM 1.5G irradiation from a Xe arc lamp with an AM 1.5 global filter. Simulator irradiance was characterized using a calibrated spectrometer and illumination intensity was set using an NREL certified silicon diode with an integrated KG1 optical filter. Spectral mismatch factors were calculated for each device in this report to be less than 10%; this value is slightly higher than is typical due to the spectral lines of Xe between 800 and 1000 nm. J/V curves shown are uncorrected for the mismatch, but the presented EQE curves integrated with respect to the solar spectrum match the presented short circuit current to within 10% and the irradiance profile of the simulator to within 5%. Quantum efficiency measurements were made with a Xe lamp, monochromator, optical chopper, and lock-in amplifier and photon flux was determined by a calibrated silicon photodiode. To maximize the ease and accuracy of the EQE calibration, the monochromatic beam was focused into a spot significantly smaller than the device area using a reflective microscope objective and thus 100% of the photons in the beam were assumed to be incident on the device.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A polymer of the formula: 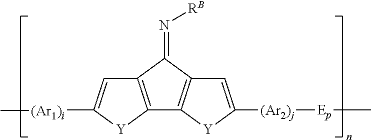
wherein R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl;
E_{P} is an electron-poor or electron-deficient aromatic moiety;
i is an integer independently selected from 0, 1, or 2;
j is an integer independently selected from 0, 1, or 2;
each Ar₁ and each Ar₂ are independently selected from unsubstituted C₆-C₂₀ aryl, substituted C₆-C₂₀ aryl, unsubstituted C₃-C₂₀ heteroaryl, and substituted C₃-C₂₀ heteroaryl;
n is an integer of at least about 5; and
Y is selected from the group consisting of S, —CH═CH—, Se, NH, NR¹ or Si, wherein R¹ is selected from C₁-C₂₄ hydrocarbyl.

2. The polymer of claim 1, wherein i is 1, j is 1, each Ar₁ is thiophene, and each Ar₂ is thiophene.

3. A polymer of the formula: 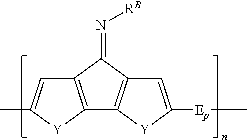
wherein:
R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl;
E_{P} is an electron-poor aromatic moiety;
n is an integer of at least about 5; and
Y is selected from the group consisting of S, —CH═CH—, Se, NH, NR¹ or Si, wherein R¹ is selected from C₁-C₂₄ hydrocarbyl;
wherein the polymer can be terminated at its ends with a C₀-C₂₄ hydrocarbyl group.

4. The polymer of claim 1, wherein E_{P} is selected from substituted and unsubstituted moieties selected from the group consisting of thiadiazoloquinoxaline, quinoxaline, thienothiadiazole, thienopyridine, thienopyrazine, pyrazinoquinoxaline, benzothiadiazole, bis-benzothiadiazole, benzobisthiadiazole, thiazole, thiadiazolothienopyrazine, and diketopyrrolopyrrole.

5. The polymer of claim 1, wherein E_{P} is substituted with one or more C₁-C₂₄ hydrocarbyl groups or —O—C₁-C₂₄ hydrocarbyl groups.

6. The polymer of claim 1, wherein the substituted R^{B} moieties are substituted with one or more substituents selected from the group consisting of F, Cl, Br, I, halogen, —R², —OH, —OR², —COOH, —COOR², —NH₂, —NHR², or NR²R³, where R² and R³ are independently selected from a C₁-C₂₄ hydrocarbyl group.

7. The polymer of claim 6, wherein each R^{B} moiety is selected from the group consisting of

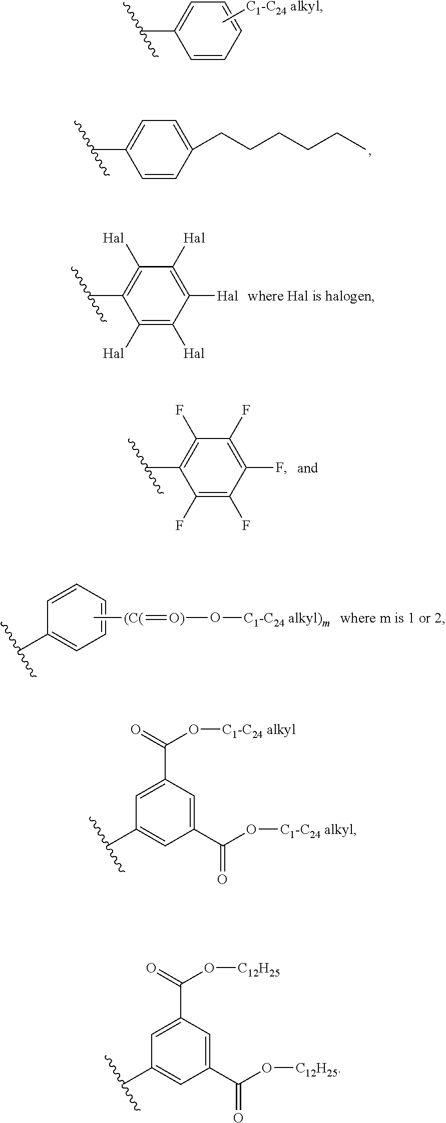

8. The polymer of claim 1, wherein each E_{P} moiety is selected from the group consisting of

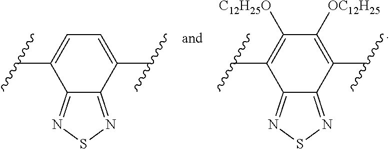

9. The polymer of claim 1, wherein each R^{B} group on the polymer is identical.

10. The polymer of claim 1, wherein Y is S.

11. The polymer of claim 1, wherein Y is —CH═CH—.

12. A compound of the formula: 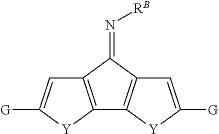
wherein:
R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl;
Y is selected from the group consisting of S, —CH═CH—, Se, NH, NR¹ or Si, wherein R¹ is selected from C₁-C₂₄ hydrocarbyl; and
G is a leaving group.

13. The compound of claim 12, wherein G is a leaving group suitable for a Stille-type polymerization reaction, a Suzuki-type polymerization reaction, or a Yamamoto-type polymerization reaction.

14. The compound of claim 12, wherein G is selected from the group consisting of Br, Cl, I, triflate (trifluoromethanesulfonate), a trialkyl tin compound, boronic acid (—B(OH)₂), or a boronate ester (—B(OR₄)₂, where each R₄ is C₁-C₁₂ alkyl or the two R₄ groups combine to form a cyclic boronic ester of the form

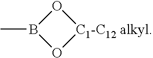

15. The compound of claim 12, wherein G is (CH₃)₃—Sn—.

16. The compound of claim 12, wherein G is

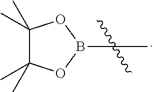 [SMILES: *B1OC(C)(C)C(C)(C)O1 ; "."]

17. The compound of claim 12, wherein G is Br.

18. The compound of claim 12, wherein Y is S.

19. The compound of claim 12, wherein Y is —CH═CH—.

20. A compound of the formula: 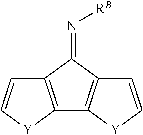
wherein:
R^{B} is selected from unsubstituted C₁-C₃₆ hydrocarbyl, substituted C₁-C₃₆ hydrocarbyl, unsubstituted C₆-C₂₀ aryl, substituted C₆-C₂₀ aryl, unsubstituted C₃-C₂₀ heteroaryl, substituted C₃-C₂₀ heteroaryl, unsubstituted —C₀-C₃₆ hydrocarbylene-C₆-C₂₀ aryl-C₀-C₃₆ hydrocarbyl, and substituted —C₀-C₃₆ hydrocarbylene-C₆-C₂₀ aryl-C₀-C₃₆ hydrocarbyl; and
Y is selected from the group consisting of S, —CH═CH—, Se, NH, NR¹ or Si, wherein R¹ is selected from C₁-C₂₄ hydrocarbyl.

21. A device comprising:
a) a first hole-collecting electrode, optionally coated onto a transparent substrate;
b) an optional hole-transporting layer adjacent to the first electrode;
c) a bulk heterojunction layer (BHJ layer) comprising a polymer of claim 1 and an electron acceptor;
d) an optional hole-blocking, exciton-blocking, or electron-transporting layer; and
e) a second electron-collecting electrode.
